(19) United States
(12) Patent Application Publication
(10) Pub. No.: US 2022/0282339 A1
(43) Pub. Date: Sep. 8, 2022

(54) GENETIC PURITY ESTIMATE METHOD BY SEQUENCING

(71) Applicant: INDIANA CROP IMPROVEMENT ASSOCIATION, LAFAYETTE, IN (US)

(72) Inventors: SRILAKSHMI MAKKENA, Carmel, IN (US); Md. Shofiqul Islam, West Lafayette, IN (US); Matheus Romanos Benatti, West Lafayette, IN (US); Peizhong Zheng, Carmel, IN (US)

(21) Appl. No.: 17/652,963

(22) Filed: Mar. 1, 2022

Related U.S. Application Data
(60) Provisional application No. 63/200,338, filed on Mar. 2, 2021.

(51) Int. Cl. C12Q 1/6895; C12Q 1/6811; C12Q 1/6851

(52) U.S. Cl. CPC C12Q 1/6895; C12Q 1/6811; C12Q 1/6851; C12Q 2600/156; C12Q 2600/13

(57) ABSTRACT

The method described presents a novel method of quantitative estimation of genetic quality of crop for a specific trait using pyrosequencing and next generation sequencing. The method quantitatively estimates the presence of a seed lot with seed of unwanted genetic trait using the allele frequency. The method assesses the genetic purity of a trait quantitatively based on allele frequency of the genetic variation between the desired and the contaminant's locus. Allele frequency is obtained by sequencing amplicons with a sequencing primer binding at the intersection of the site of genetic variation that differentiates between contaminant and the desired trait. The true genetic purity of an unknown seed lot is estimated by substituting the allele frequency value in a regression equation derived from the allele frequencies of several standards used in every sequencing experiment.

### CROSS-REFERENCE TO RELATED APPLICATIONS

This application claims priority under 35 U.S.C. § 119 to provisional patent application U.S. Ser. No. 63/200,338 filed Mar. 2, 2021. The provisional patent application is herein incorporated by reference in its entirety, including without limitation, the specification, claims, and abstract, as well as any figures, tables, appendices, or drawings thereof.

### BACKGROUND

Genetic quality information of seed stock is vital for product development, product commercialization, commercial seed production, and marketing of seed. Genetic quality testing of crop and seed stock is necessary to ensure that the crop grown in the field for a variety of uses, including grazing, plant parts for use as raw materials (e.g., Roots, stems, leaves, flowers and flower parts) and seed supplied to crop growers has specified genetic traits. Further, trait genetic information is used for monitoring food materials originated from crops improved through genetic modification (GM) and gene editing (GE) technologies throughout the food supply chain in order to meet labelling and regulatory requirements that are in place in specific geographic regions. For seed certification and quality assurance according to the standards set by seed certification agencies, every seed lot sold for growing crops must meet the minimum genetic purity requirements and the genetic quality information must be specified on the certification tag which may include specification of genetic trait and genetic purity of the trait. The quantitative expression of percent seed with genetic trait in a seed lot is called as seed genetic purity.

Currently, the genetic quality of a given seed lot is determined by testing a representative seed sample drawn from that seed lot in three different ways: 1. Phenotypically, by growing seed into plants (Grow Out test) and visual examination for specific traits (flower color, growth habit, tassels etc.) and herbicide tolerance of GM traits is tested by spraying with herbicides (bio-assay); 2. Genotypically, by testing DNA from the seed for the presence or absence of a specific DNA sequence of the gene associated with the trait, using Polymerase Chain Reaction (PCR), Real-Time PCR (RT-PCR)(Holst-Jensen et al., 2003), DNA Fingerprints and Southern Blot; 3. Biochemically, using isozyme electrophoresis, by testing protein fingerprints of total seed protein using Iso Electric Focusing (IEF) and SDS-PAGE and/or by looking for the expression of a trait specific protein using Western Blot, ELISA and Lateral Flow Strip methods for GM traits (Smith & Register III, 1998) Except the real-time RT-PCR method, all other diagnostic methods test individual seed of 30 to 400 for each seed lot.

The method used and the requirements for genetic quality testing of seeds and/or traits depends on the genetic nature of the trait and the breeding method used for crop improvement. Genetic traits could be classified into three types based on the source of genetic variation. These include, 1. Native traits: natural source of genes/genetic variation present in a plant species is used to improve crops; 2. Transgenic traits/Genetically Modified Organisms: a gene from one organism is purposely moved to improve another organism; 3. Gene Edited traits: a plant's DNA sequence at a specific location is changed by removing, adding or altering DNA sequences. Every genetic trait has a unique DNA sequence and the variations in the gene sequence (genetic variations), including single nucleotide variation, either insertion or deletion of a few base pairs or an entire gene (natural variation or GE traits), and introduction of an entirely new gene sequence (GM traits) information is used for determining the genetic quality of a trait.

The choice and/or the combination of methods used vary from product development to commercialization phase since the depth of genetic information required about a seed lot and objectives of the quality control program are different at both phases. During the product development phase, genetic information of seed is used for selecting diverse parents, parentage verification, genetic identity, genetic homogeneity of the seed material and genetic purity of specific traits whereas for product commercialization and marketing, genetic information about the seed lot genetic purity, trait purity and parentage verification of hybrid seed are important (Gowda et al., n.d.). Minimum number of seed that need to be tested for a seed lot depends on the regulatory requirements and further depends on the genetic nature of the trait and if the seed is a hybrid or a variety. A statistical tool called SeedCalc was developed for designing seed testing plans for purity/impurity characteristics including testing for adventitious presence levels of GM traits in conventional seed lots. This application can also be used to estimate purity or impurity in a seed lot (Laffont et al., 2005; Remund et al., 2001). Depending on the diagnostic method employed, information about various quality parameters of a seed lot will be obtained. Trait genetic purity for a seed lot is obtained by testing trait specific markers.

Though there are a variety of diagnostic methods available for addressing a specific challenge, these methods have some limitations. Phenotypic examination of traits is expensive and takes a long time to grow plants up to a desired stage for the visual expression of traits and its use is further limited by shorter growing season in certain geographic locations. For assessing the genetic quality of a seed lot, at least 100-400 seeds must be tested to meet the certification standards and DNA-based methods, PCR, DNA Fingerprinting and Southern Blot and biochemical methods are all qualitative methods, provide only a presence/absence information when applied on bulk seed and they become expensive to test individual seed. The limitations of time and growing season associated with the phenotype-based purity estimation and testing cost associated with the DNA-based methods have been considerably reduced by applying quantitative RT-PCR. However, the applications of RT-PCR method for quantitative assessment of trait genetic quality is limited due to its specific requirements for good quality input DNA, detection probe, and assay standardization for several factors for achieving reliable detection (Cankar et al., 2006; Demeke & Jenkins, 2010). Further, it cannot reliably detect single nucleotide polymorphism (SNP) and small insertion and deletion genetic variations and the range of accuracy of assessment of trait genetic purity quantitatively is narrow.

Array based genotyping technologies for SNP genetic variation detection are being used for determining the identity and homogeneity of a seed lot (Chen J, 2016). In the array-based technologies, DNA from seeds of 5-10 is tested for each lot and the number of SNPs tested varies based on the objective of the quality control testing requirements. The qualitative information obtained from 5-10 seeds is used for determining the genetic quality of a seed lot. Though the array technologies are cheaper and faster, it becomes expensive to test 400-3000 seed to meet the certification requirements. Hypothetically, to address the challenges associated with other methods, including reliability of detection of a variety of genetic variations associated with a specific genomic locus and/or loci, accuracy and sample throughput, any next generation sequencing (NGS) technology when combined with data analytics that could calculate the allele frequency information of either a specific locus or loci and further statistical analysis to draw meaningful conclusions about the seed lot could be used.

A patent application WO PCT/EU2019/070386 demonstrates the application of NGS technology for assessing genetic purity of seed lot. The method estimates the quantitative value of genetic purity of a seed lot using the qualitative information obtained from several sub-samples. Seed sample was divided into several sub-samples (16-24 sub-samples) and each sub-sample is tested for the qualitative information of presence or absence of a contaminant using the allele frequency of marker loci. Seventeen marker loci were tested for each sub-sample and a qualitative score of presence or absence of contaminant was assigned when at least 3 loci were detected to have alternative allele based on allele frequency of tested loci. Qualitative profile of sub-samples was used for calculating the quantitative value using the Seed Calc software (Remund, 2001). Molecular profile information obtained using this approach will be valuable for determining the genetic identity/conformity of the variety tested and for detecting the possible contaminant. Though the method was proved to be valuable for identifying contaminant at 1% level, there was no further information on its application for detecting higher levels of contamination. Further, this approach becomes expensive and has a longer turnaround time. Another problem associated with using NGS technologies is their requirement for a dedicated data analysis pipeline. Availability of any high throughput, cost-effective trait genetic quality assessment method with a fast turnaround time is required and would be valuable for seed industry and for the regulation of foods with gene edited traits in the food supply chain.

### SUMMARY

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

The method presented here relates to the quantitative assessment of trait genetic purity of a seed lot using pyrosequencing. Pyrosequencing is a real-time quantitative bioluminescence technique used for DNA sequencing that can detect and quantify the relative levels or frequency of genetic variants, specifically, SNP and few base pairs of insertion/deletion (indel) genetic variations in a DNA sequence.

Pyrosequencing has been used for detection of genetic variation for a variety of applications. In clinical genetic diagnostics, pyrosequencing is routinely used in detecting and quantifying oncogene specific marker genetic variations (El-Deiry et al., 2019). (Tsiatis et al., 2010) reported that there was no false positive or false negative detection of KRAS oncogene marker variation using Pyrosequencing method.

In the patent application CN102358911A, pyrosequencing has been used to improve the efficiency of hybridity testing of corn, cucumber and rice seed. The method uses DNA extracted from either three seed or three leaf tissue bulks to check if the DNA from pool of three seed scores an allele frequency of 0.5 for SNP and indel genetic variations. In patent Ser. No. 10/928,766 A, pyrosequencing was used for hybridity verification testing of cucumber seed for confirming the allele frequency of 0.5 by testing DNA extracted from a pool of 150 seed.

Pyrosequencing was proposed as a detection method for transgenic event detection in corn and Brassica (U.S. Pat. No. 7,897,342 B2 and U.S. Pat. No. 8,993,238 B2). (Song et al., 2014) have used pyrosequencing on a portable photodiode-based bioluminescence sequencer for detecting genetically modified organisms (GMO) or transgenic events in corn and soybean. Pyrosequencing was used to quantify incidence of a specific Aspergillus flavus strain within a complex of fungal community applied as a seed treatment on commercial cotton seed (Das et al., 2008). Patent number CN104419755A is related to the use of Pyrosequencing for detecting and quantifying the adulteration of Japanese honey suckle, an ingredient used in Chinese patented medicines, health products and foods with Lonicera confusa by quantifying a SNP genetic variation that differentiates the ingredient and the adulterant.

There are a number of publicly available tools to help choose and/or design target sequences as well as lists of bioinformatically determined unique sgRNAs for different genes in different species such as, but not limited to, the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

To use the CRISPR system, both sgRNA and a Cas endonuclease (e.g., Cas9) should be expressed or present (e.g., as a ribonucleoprotein complex) in a target cell. The insertion vector can contain both cassettes on a single plasmid or the cassettes are expressed from two separate plasmids. CRISPR plasmids are commercially available such as the px330 plasmid from Addgene (75 Sidney St, Suite 550A•Cambridge, Mass. 02139). Use of clustered regularly interspaced short palindromic repeats (CRISPR)-associated (Cas)-guide RNA technology and a Cas endonuclease for modifying plant genomes are also at least disclosed by Svitashev et al., 2015, Plant Physiology, 169 (2): 931-945; Kumar and Jain, 2015, J Exp Bot 66: 47-57; and in U.S. Patent Application Publication No. 20150082478, which is specifically incorporated herein by reference in its entirety. Cas endonucleases that can be used to effect DNA editing with sgRNA include, but are not limited to, Cas9, Cpf1 (Zetsche et al., 2015, Cell. 163(3):759-71), C2c1, C2c2, C2c3, cms1 (Shmakov et al., Mol Cell. 2015 Nov. 5; 60(3):385-97) and Cas 13A/B (Barrangoul et al., 2017, Molecular cell, 65: 582-584; Abudayyeh et al., 2017, Nature 550: 280-284). The Cas 13 A ORB (Cas 13A/B) can recognize and cleave RNA, not DNA. this could be applied when RNA-degradation (RNAI-like) is desired.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, introduced into the cells, and positive selection is performed to isolate homologous recombination events. The DNA carrying the homologous sequence can be provided as a plasmid, single or double stranded oligo. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach but requires the use of two different targeting constructs. In the first step, a standard targeting vector with 3′ and 5′ homology arms is used to insert a dual positive/negative selectable cassette near the location where the mutation is to be introduced. After the system component have been introduced to the cell and positive selection applied, HR events could be identified. Next, a second targeting vector that contains a region of homology with the desired mutation is introduced into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and re-ligation within the spacer region. The staggered DNA cuts made by Cre in the spacer region are separated by 6 base pairs to give an overlap region that acts as a homology sensor to ensure that only recombination sites having the same overlap region recombine.

The site specific recombinase system offers means for the removal of selection cassettes after homologous recombination events. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombination events that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Chemical mutagenesis provides an inexpensive and straightforward way to generate a high density of novel nucleotide diversity in the genomes of plants and animals. Mutagenesis therefore can be used for functional genomic studies and also for plant breeding. The most commonly used chemical mutagen in plants is ethyl methanesulfonate (EMS). EMS has been shown to induce primarily single base point mutations. Hundreds to thousands of heritable mutations can be induced in a single plant line. A relatively small number of plants, therefore, are needed to produce populations harboring deleterious alleles in most genes. EMS mutagenized plant populations can be screened phenotypically (forward-genetics), or mutations in genes can be identified in advance of phenotypic characterization (reverse-genetics). Reverse-genetics using chemically induced mutations is known as Targeting Induced Local Lesions IN Genomes (TILLING) (see, for example, Jankowicz-Cieslak, J, Till, B. Chemical Mutagenesis of Seed and Vegetatively Propagated Plants Using EMS, Current Protocols in Plant Biology, 1:4 pp. 617-635).

Genome engineering includes altering the genome by deleting, inserting, mutating, or substituting specific nucleic acid sequences. The alteration can be gene- or location-specific. Genome engineering can use site-directed nucleases, such as Cas proteins and their cognate polynucleotides, to cut DNA, thereby generating a site for alteration. In certain cases, the cleavage can introduce a double-strand break (DSB) in the DNA target sequence. DSBs can be repaired, e.g., by non-homologous end joining (NHEJ), microhomology-mediated end joining (MMEJ), or homology-directed repair (HDR). HDR relies on the presence of a template for repair. In some examples of genome engineering, a donor polynucleotide or portion thereof can be inserted into the break.

Clustered regularly interspaced short palindromic repeats (CRISPR) and CRISPR-associated proteins (Cas) constitute the CRISPR-Cas system. The CRISPR-Cas system provides adaptive immunity against foreign DNA in bacteria (see, e.g., Barrangou, R., et al., Science 315:1709-1712 (2007); Makarova, K. S., et al., Nature Reviews Microbiology 9:467-477 (2011); Garneau, J. E., et al., Nature 468:67-71 (2010); Sapranauskas, R., et al., Nucleic Acids Research 39:9275-9282 (2011)).

CRISPR-Cas systems have recently been reclassified into two classes, comprising five types and sixteen subtypes (see Makarova, K., et al., Nature Reviews Microbiology 13:1-15 (2015)). This classification is based upon identifying all Cas genes in a CRISPR-Cas locus and determining the signature genes in each CRISPR-Cas locus, ultimately placing the CRISPR-Cas systems in either Class 1 or Class 2 based upon the genes encoding the effector module, i.e., the proteins involved in the interference stage. Recently a sixth CRISPR-Cas system (Type VI) has been identified (see Abudayyeh O., et al., Science 353(6299):aaf5573 (2016)). Certain bacteria possess more than one type of CRISPR-Cas system.

Class 1 systems have a multi-subunit crRNA-effector complex, whereas Class 2 systems have a single protein, such as Cas9, Cpf1, C2c1, C2c2, C2c3, or a crRNA-effector complex. Class 1 systems comprise Type I, Type III, and Type IV systems. Class 2 systems comprise Type II, Type V, and Type VI systems.

Type II systems have cas1, cas2, and cas9 genes. The cas9 gene encodes a multi-domain protein that combines the functions of the crRNA-effector complex with DNA target sequence cleavage. Type II systems are further divided into three subtypes, subtypes II-A, II-B, and II-C. Subtype II-A contains an additional gene, csn2. Examples of organisms with a subtype II-A systems include, but are not limited to, Streptococcus pyogenes, Streptococcus thermophilus, and Staphylococcus aureus. Subtype II-B lacks the csn2 protein but has the cas4 protein. An example of an organism with a subtype II-B system is Legionella pneumophila. Subtype II-C is the most common Type II system found in bacteria and has only three proteins, Cas1, Cas2, and Cas9. An example of an organism with a subtype II-C system is Neisseria lactamica.

Type V systems have a cpf1 gene and cas1 and cas2 genes (see Zetsche, B., et al., Cell 163:1-13 (2015)). The cpf1 gene encodes a protein, Cpf1, that has a RuvC-like nuclease domain that is homologous to the respective domain of Cas9 but lacks the HNH nuclease domain that is present in Cas9 proteins. Type V systems have been identified in several bacteria including, but not limited to, Parcubacteria bacterium, Lachnospiraceae bacterium, Butyrivibrio proteoclasticus, Peregrinibacteria bacterium, Acidaminococcus spp., Porphyromonas macacae, Porphyromonas crevioricanis, Prevotella disiens, Moraxella bovoculi, Smithella spp., Leptospira inadai, Franciscella tularensis, Franciscella novicida, Candidatus methanoplasma termitum, and Eubacterium eligens. Recently it has been demonstrated that Cpf1 also has RNase activity and is responsible for pre-crRNA processing (see Fonfara, I., et al., Nature 532(7600):517-521 (2016)).

In Class 2 systems, the crRNA is associated with a single protein and achieves interference by combining nuclease activity with RNA-binding domains and base-pair formation between the crRNA and a nucleic acid target sequence.

In Type II systems, nucleic acid target sequence binding involves Cas9 and the crRNA, as does nucleic acid target sequence cleavage. In Type II systems, the RuvC-like nuclease (RNase H fold) domain and the HNH (McrA-like) nuclease domain of Cas9 each cleave one of the strands of the double-stranded nucleic acid target sequence. The Cas9 cleavage activity of Type II systems also requires hybridization of crRNA to a tracrRNA to form a duplex that facilitates the crRNA and nucleic acid target sequence binding by the Cas9 protein. The RNA-guided Cas9 endonuclease has been widely used for programmable genome editing in a variety of organisms and model systems (see, e.g., Jinek M., et al., Science 337:816-821 (2012); Jinek M., et al., eLife 2:e00471. doi: 10.7554/eLife.00471 (2013); U.S. Published Patent Application No. 2014-0068797, published 6 Mar. 2014).

In Type V systems, nucleic acid target sequence binding involves Cpf1 and the crRNA, as does nucleic acid target sequence cleavage. In Type V systems, the RuvC-like nuclease domain of Cpf1 cleaves one strand of the double-stranded nucleic acid target sequence, and a putative nuclease domain cleaves the other strand of the double-stranded nucleic acid target sequence in a staggered configuration, producing 5′ overhangs, which is in contrast to the blunt ends generated by Cas9 cleavage.

The Cpf1 cleavage activity of Type V systems does not require hybridization of crRNA to tracrRNA to form a duplex, rather the crRNA of Type V systems uses a single crRNA that has a stem-loop structure forming an internal duplex. Cpf1 binds the crRNA in a sequence and structure specific manner that recognizes the stem loop and sequences adjacent to the stem loop, most notably the nucleotides 5′ of the spacer sequences that hybridizes to the nucleic acid target sequence. This stem-loop structure is typically in the range of 15 to 19 nucleotides in length. Substitutions that disrupt this stem-loop duplex abolish cleavage activity, whereas other substitutions that do not disrupt the stem-loop duplex do not abolish cleavage activity. Nucleotides 5′ of the stem loop adopt a pseudo-knot structure further stabilizing the stem-loop structure with non-canonical Watson-Crick base pairing, triplex interaction, and reverse Hoogsteen base pairing (see Yamano, T., et al., Cell 165(4):949-962 (2016)). In Type V systems, the crRNA forms a stem-loop structure at the 5′ end, and the sequence at the 3′ end is complementary to a sequence in a nucleic acid target sequence.

Other proteins associated with Type V crRNA and nucleic acid target sequence binding and cleavage include Class 2 candidate 1 (C2c1) and Class 2 candidate 3 (C2c3). C2c1 and C2c3 proteins are similar in length to Cas9 and Cpf1 proteins, ranging from approximately 1,100 amino acids to approximately 1,500 amino acids. C2c1 and C2c3 proteins also contain RuvC-like nuclease domains and have an architecture similar to Cpf1. C2c1 proteins are similar to Cas9 proteins in requiring a crRNA and a tracrRNA for nucleic acid target sequence binding and cleavage but have an optimal cleavage temperature of 50.degree. C. C2c1 proteins target an AT-rich protospacer adjacent motif (PAM), similar to the PAM of Cpf1, which is 5′ of the nucleic acid target sequence (see, e.g., Shmakov, S., et al., Molecular Cell 60(3):385-397 (2015)).

Class 2 candidate 2 (C2c2) does not share sequence similarity with other CRISPR effector proteins and was recently identified as a Type VI system (see Abudayyeh, O., et al., Science 353(6299):aaf5573 (2016)). C2c2 proteins have two HEPN domains and demonstrate single-stranded RNA cleavage activity. C2c2 proteins are similar to Cpf1 proteins in requiring a crRNA for nucleic acid target sequence binding and cleavage, although not requiring tracrRNA. Also, similar to Cpf1, the crRNA for C2c2 proteins forms a stable hairpin, or stem-loop structure, that aids in association with the C2c2 protein. Type VI systems have a single polypeptide RNA endonuclease that utilizes a single crRNA to direct site-specific cleavage. Additionally, after hybridizing to the target RNA complementary to the spacer, C2c2 becomes a promiscuous RNA endonuclease exhibiting non-specific endonuclease activity toward any single-stranded RNA in a sequence independent manner (see East-Seletsky, A., et al., Nature 538(7624):270-273 (2016)).

Regarding Class 2 Type II CRISPR-Cas systems, a large number of Cas9 orthologs are known in the art as well as their associated polynucleotide components (tracrRNA and crRNA) (see, e.g., Fonfara, I., et al., Nucleic Acids Research 42(4):2577-2590 (2014), including all Supplemental Data; Chylinski K., et al., Nucleic Acids Research 42(10):6091-6105 (2014), including all Supplemental Data). In addition, Cas9-like synthetic proteins are known in the art (see U.S. Published Patent Application No. 2014-0315985, published 23 Oct. 2014).

Cas9 is an exemplary Type II CRISPR Cas protein. Cas9 is an endonuclease that can be programmed by the tracrRNA/crRNA to cleave, in a site-specific manner, a DNA target sequence using two distinct endonuclease domains (HNH and RuvC/RNase H-like domains) (see U.S. Published Patent Application No. 2014-0068797, published 6 Mar. 2014; see also Jinek, M., et al., Science 337:816-821 (2012)).

Typically, each wild-type CRISPR-Cas9 system includes a crRNA and a tracrRNA. The crRNA has a region of complementarity to a potential DNA target sequence and a second region that forms base-pair hydrogen bonds with the tracrRNA to form a secondary structure, typically to form at least one stem structure. The region of complementarity to the DNA target sequence is the spacer. The tracrRNA and a crRNA interact through a number of base-pair hydrogen bonds to form secondary RNA structures. Complex formation between tracrRNA/crRNA and Cas9 protein results in conformational change of the Cas9 protein that facilitates binding to DNA, endonuclease activities of the Cas9 protein, and crRNA-guided site-specific DNA cleavage by the endonuclease Cas9. For a Cas9 protein/tracrRNA/crRNA complex to cleave a double-stranded DNA target sequence, the DNA target sequence is adjacent to a cognate PAM. By engineering a crRNA to have an appropriate spacer sequence, the complex can be targeted to cleave at a locus of interest, e.g., a locus at which sequence modification is desired.

A variety of Type II CRISPR-Cas system crRNA and tracrRNA sequences, as well as predicted secondary structures are known in the art (see, e.g., Ran, F. A., et al., Nature 520(7546):186-191 (2015), including all Supplemental Data, in particular Extended Data FIG. 1; Fonfara, I., et al., Nucleic Acids Research 42(4):2577-2590 (2014), including all Supplemental Data, in particular Supplemental Figure S 11).

The spacer of Class 2 CRISPR-Cas systems can hybridize to a nucleic acid target sequence that is located 5′ or 3′ of a PAM, depending upon the Cas protein to be used. A PAM can vary depending upon the Cas polypeptide to be used. For example, if Cas9 from S. pyogenes is used, the PAM can be a sequence in the nucleic acid target sequence that comprises the sequence 5′-NRR-3′, wherein R can be either A or G, N is any nucleotide, and N is immediately 3′ of the nucleic acid target sequence targeted by the nucleic acid target binding sequence. A Cas protein may be modified such that a PAM may be different compared with a PAM for an unmodified Cas protein. If, for example, Cas9 from S. pyogenes is used, the Cas9 protein may be modified such that the PAM no longer comprises the sequence 5′-NRR-3′, but instead comprises the sequence 5′-NNR-3′, wherein R can be either A or G, N is any nucleotide, and N is immediately 3′ of the nucleic acid target sequence targeted by the nucleic acid target sequence.

Other Cas proteins recognize other PAMs, and one of skill in the art is able to determine the PAM for any particular Cas protein. For example, Cpf1 has a thymine-rich PAM site that targets, for example, a TTTN sequence (see Fagerlund, R., et al., Genome Biology 16:251 (2015)).

Off-target effects stemming from CRISPR/Cas9 off-target cleavage has increasingly become a potential limitation for therapeutic uses. For example, the type II CRISPR system, which is derived from S. pyogenes, is reconstituted in mammalian cells using Cas9, a specificity-determining CRISPR RNA (cfRNA) and an auxiliary trans-activating RNA (tracrRNA). The term “off target effect” broadly refers to any impact (frequently adverse) distinct from and not intended as a result of the on-target treatment or procedure. The crRNA and tracrRNA duplexes can be fused to generate a single-guide RNA (sgRNA). The first 20 nucleotides of the sgRNA are complementary to the target DNA sequence, and those 20 nucleotides are followed by the protospacer adjacent motif (PAM).

The present invention includes a method for testing the genetic quality of crop/seed lot for a specific trait wherein the crop/plant may be maize (Zea mays), soybean (Glycine max), cotton (Gossypium hirsutum), peanut (Arachis hypogaea), barley (Hordeum vulgare); oats (Avena sativa); orchard grass (Dactylis glomerata); rice (Oryza sativa, including indica and Japonica varieties); Sorghum (Sorghum bicolor); sugar cane (Saccharum sp); tall fescue (Festuca arundinacea); turfgrass species (e.g. species: Agrostis stolonifera, Poa pratensis, Stenotaphrum secundatum); wheat (Triticum aestivum), and alfalfa (Medicago sativa), members of the genus Brassica, broccoli, cabbage, carrot, cauliflower, Chinese cabbage, cucumber, dry bean, eggplant, fennel, garden beans, gourd, leek, lettuce, melon, okra, onion, pea, pepper, pumpkin, radish, spinach, squash, sweet corn, tomato, watermelon, ornamental plants, and other fruit, vegetable, tuber, oilseed, and root crops, wherein oilseed crops include soybean, canola, oil seed rape, oil palm, sunflower, olive, corn, cottonseed, peanut, flaxseed, safflower, and coconut, and where traits comprising at least one sequence of interest, further defined as conferring a preferred property selected from the group consisting of herbicide tolerance, disease resistance, insect or pest resistance, altered fatty acid, protein or carbohydrate metabolism, increased grain yield, increased oil, increased nutritional content, increased growth rates, enhanced stress tolerance, preferred maturity, enhanced organoleptic properties, altered morphological characteristics, other agronomic traits, traits for industrial uses, or traits for improved consumer appeal, wherein said traits may be nontransgenic or transgenic.

Transposable elements (TEs) are DNA segments capable of changing their position in the genome. In plants, TEs occupy a significant portion of genomes and, upon mobilization, are capable of driving dynamic changes through the formation of novel structural variants. These can range from simple insertional polymorphisms, resulting in gene knockouts, to complex rearrangements with profound effects on gene evolution, dosage, and regulation, ultimately resulting in phenotypic diversity.

Abiotic stresses, such as low or high temperature, deficient or excessive water, high salinity, heavy metals, and ultraviolet radiation, are hostile to plant growth and development, leading to great crop yield penalty worldwide. It is getting imperative to equip crops with multistress tolerance to relieve the pressure of environmental changes and to meet the demand of population growth, as different abiotic stresses usually arise together in the field. The feasibility is raised as land plants actually have established more generalized defenses against abiotic stresses, including the cuticle outside plants, together with unsaturated fatty acids, reactive species scavengers, molecular chaperones, and compatible solutes inside cells. In stress response, they are orchestrated by a complex regulatory network involving upstream signaling molecules including stress hormones, reactive oxygen species, gasotransmitters, polyamines, phytochromes, and calcium, as well as downstream gene regulation factors, particularly transcription factors. (He, et. al. Front. Plant Sci., Vol. 9, 7 Dec. 2018).

The hemp plant produces cannabinoids such as THC and cannabidiol (CBD, a non-psychoactive compound that has been shown to have certain therapeutic properties) in hair-like structures called trichomes that are found in the flowers and, to a lesser extent, the leaves. However, very little THC and CBD are found in the plant in its natural state. Instead, the acid form of each (THC-A and CBD-A) is produced, which can then be transformed by the removal of a carboxyl group and the subsequent release of a molecule of carbon dioxide. This process of decarboxylation occurs over time or with heat.

The legal definition of hemp was spelled out in Section 7606 of the 2014 Farm Bill, “The term ‘industrial hemp” means the plant Cannabis sativa L. and any part of such plant, whether growing or not, with a delta-9 tetrahydrocannabinol concentration of not more than 0.3% on a dry weight basis.”

Section 297A under Subtitle G of the 2018 Farm Bill includes similar language, “The term ‘hemp’ means the plant Cannabis sativa L. and any part of that plant, including the seeds thereof and all derivatives, extracts, cannabinoids, isomers, acids, salts and salts of isomers, whether growing or not, with a delta-9 tetrahydrocannabinol concentration of not more than 0.3% on a dry weight basis.”

The 2014 Farm Bill cleared the way for research to be conducted with hemp by institutions of higher education or state departments of agriculture. The 2018 Farm Bill further legalized the commercialization of hemp. The key to working with the crop is ensuring that the concentration of delta-9 tetrahydrocannabinol (THC), the psychoactive chemical found in marijuana in relatively high concentrations, remains below the 0.3% threshold. The testing method of the instant invention may be used for this purpose.

### DESCRIPTION OF THE FIGURES

The drawings are presented for exemplary purposes and may not be to scale unless otherwise indicated.

FIG. 1. Standard curve analysis for validating the amplification efficiency of three different primer pairs on RT-PCR; primer pair 1=CYP79A1F+CYP79R2; primer pair 2=CYP79A1F+CYP79R3; Primer pair 3=CYP79A1F2+CYP79R2 at 100, 10, 1, 0.1 and 0.01 ng of genomic DNA template from wild type seed.

FIG. 2. Fluorescence was detected in PCR amplification from dhurrin free sorghum (WL75) DNA. The amplification plot shows the fluorescence from 75 nanograms of wild type (WT75) and dhurrin free (WL75) DNA.

FIG. 3. Standard curve analysis for validating the amplification efficiency of primer pair CYP79A1ASPFR1 and CP79A1RASP1 on RT-PCR with detection probe, CYP79Probe 2 at 100, 10, 1, 0.1 and 0.01 ng of genomic DNA template from wild type seed.

FIG. 4. Regression equation was derived using the pyrosequencer estimated allele quantification values for the standards. The standards are the DNA extracted from spiked seed samples. Spiked seed samples were prepared by mixing known quantities of wild type seed (with no DF trait) to seed sample with dhurrin free trait. Spiked standards used were 0.1%, 0.2%, 0.3%, 0.5%, 1%, 2% and 5% wild type seed contamination. Regression equation was obtained by plotting pyrosequencer quantified allele frequency values from the spiked seed samples against the known spiking values (trait purity) and this regression equation was used for estimating the trait purity or level of contamination of unknown seed lots with sorghum seed consisting of wild type allele.

FIGS. 5A and B. Regression equation was derived using the pyrosequencer estimated allele quantification values for the control seed standards. The standards are the DNA extracted from spiked seed samples. Spiked seed samples were prepared by mixing known quantities of corn seed with cytoplasmic male sterile and fertile type seed. Spiked standards used were 99%, 95%, 90%, 80%, 70%, 60%, 50%, 40%, 30%, 20%, and 10% seed with sterile trait. Regression equation was obtained by plotting pyrosequencing results from the spiked seed samples against the known spiking values (trait purity) and this regression equation was used for estimating the trait purity or level of contamination of unknown seed lots.

FIG. 6. Regression equation was derived using the pyrosequencer quantified allele frequency values for the control standards made by pooling leaf punches in a known proportion, collected from seedlings of fertile and sterile cytotypes. X-axis: Male fertile cytotype specific ‘G’ allele frequency quantified by pyrosequencer. Y-axis: Genetic purity of male fertile cytotype. Standards used were 100%, 90%, 80%, 75%, 70%, 60%, 50%, 40%, 30%, 25%, 20%, 10% fertile cytotype and 100% sterile cytotypes. Regression equation was obtained by plotting pyrosequencer quantified fertile cytotype specific allele frequency values against the known spiked/trait purity values and this regression equation was used for estimating the trait purity for fertile cytotype or level of admixture of male sterile cytotypes of unknown seed lots.

FIG. 7. Linear regression equation was derived using the WideSeq estimated allele quantification values obtained from the standard samples. The standards were prepared by spiking DNA of known concentration. Linear regression equation was obtained by plotting WideSeq quantified allele frequency values from the standard samples against the known spiking values (trait purity). This linear regression equation can be used for estimating the trait purity or level of contamination of unknown seed lots with sorghum seed consisting of wild type allele when DNA are extracted from such seed lots and subjected to NextGen Sequencing using MiSeq.

### DETAILED DESCRIPTION

The present disclosure is not to be limited to that described herein. Mechanical, electrical, chemical, procedural, and/or other changes can be made without departing from the spirit and scope of the present invention. No features shown or described are essential to permit basic operation of the present invention unless otherwise indicated.

In the present invention, pyrosequencing was applied on bulked seed for detecting the adventitious presence of contaminants and trait genetic purity of a seed lot quantitatively. This method uses DNA extracted from bulked seed, amplifies DNA region surrounding the causative genetic variation followed by sequencing of amplicons using pyrosequencing technology. The method for estimating the trait genetic purity using pyrosequencing uses the below listed basic steps:

1. Identifying the genetic variation that differentiates the trait of interest from the contaminant i.e., DNA sequence of the locus associated with the trait of interest for which genetic purity needs to be quantified and identification of contaminant's genetic variation present within the same locus
2. Acquiring seed material that is pure for each of the genetic variations
3. Calculate the test weight of seed based on 1000 seed weight. Prepare seed standards by spiking pure seed of trait of interest with various proportions of contaminant seed based on 1000 seed weight. Standards of 100% pure seed for both, the seed with trait of interest and the contaminating seed must be included for every assay. If leaf punches were used, same number of uniformed leaf disks are taken from different samples. The levels of spiking can be variable depending on the genetic purity requirements for a specific trait. Make two to three replicates of seed/leaf standards.
4. To test the validity of the assay, include either blind samples prepared by an outsider
5. Extract genomic DNA from all the seed/leaf standards
6. Design primers for amplification of the genomic region surrounding the genetic variation (marker) and a sequencing primer
7. Test the primers for specificity to make sure that there are no primer dimers and amplicon is specific to the targeted region by sequencing
8. PCR amplify the marker from all standards, blind samples and any other samples tested for genetic variation using other detection methods. PCR amplification is done in two replicates for each independent DNA extraction
9. Sequence the amplicons on pyrosequencer
10. Calculate the regression equation using the known trait purity values of seed standards and the allele frequency values given by the pyrosequencer
11. Check the correlation between trait purity values of seed/leaf standards and the allele frequency values from the pyrosequencer and if r²≥0.99
12. Estimate the trait purity for unknown/blind seed/leaf sample by substituting the allele frequency of that sample in the “x” place of the derived regression equation

The method described presents a novel method of quantitative estimation of genetic quality of crop/seed lot for a specific trait using a type of DNA sequencing technology called pyrosequencing. The method quantitatively estimates the contamination/admixture/adventitious presence of a seed lot with seed of unwanted genetic trait using the allele frequency.

The method assesses the genetic purity of a trait quantitatively based on allele frequency of the genetic variation between the desired and the contaminant's locus. Allele frequency is obtained by sequencing amplicons with a sequencing primer binding at the intersection of the site of genetic variation that differentiates between contaminant and the desired trait. The true genetic purity of an unknown seed lot is estimated by substituting the allele frequency value in a regression equation derived from the allele frequencies of several standards used in every sequencing experiment.

The standards are the DNA extracted from seed mixed in various proportions of seeds with desired trait and contaminant.

The detection sensitivity or Limit of Detection (LOD) of the assay for seed lot contamination with seed of unwanted traits is 0.5% and accurately assesses the purity of a trait over a wide range of contamination with a Limit of Quantification (LOQ) of 0.5% to 99.5%. Applicability of the method across crops was verified by testing sorghum and corn seed/leaf and satisfactory results were obtained for the tested traits. In principle, this method could be applied to genetic purity testing of both native and gene edited traits with various types of genetic variation, including SNP variation, few base pair insertion and deletion variation in a bulked seed sample. Further, the methodology presented here could also be used with any next generation sequencing technology and could be customized for simultaneously testing several markers for a seed lot.

The value of the method is in the assessment of contamination over a broad range from 0.5 to 99.5%. The assay development is faster when compared to Real-Time PCR and NextGen Sequencing (NGS) methods and any laboratory providing diagnostic services to seed, and food industry can quickly adopt the method.

### Embodiments

Various embodiments of the systems and methods provided herein are included in the following non-limiting list of embodiments.

1. A method of quantitative determination of the level of a genetic trait within a seed sample by next generation sequencing comprising:
(a) acquiring at least one testing seed sample to be estimated for the level of a genetic trait of interest, a contaminant seed sample, and a pure seed sample which is pure for the genetic trait of interest;
(b) preparing seed standards by spiking the pure seed sample with various proportions of contaminant seed;
(c) extracting genomic DNA from the pure seed sample, contaminant seed sample, seed standards, and at the at least one testing seed sample;
(d) designing primers for amplification of the genomic region neighboring the genetic trait of interest and a sequencing primer;
(e) performing PCR amplification on the seed samples and seed standards using said primers;
(f) sequencing the amplicons on a next generation sequencer and calculating a regression equation using known trait purity values of seed standards and the allele frequency values given by the next generation sequencer; and
(g) calculating the estimated quantitative level of trait purity for the at least one testing seed sample using said regression equation.
2. The method of embodiment 1, wherein said genetic trait of interest comprises a polymorphism selected from the group consisting of SNPs, indels, and a variation in copy number.
3. The method of embodiment 2, wherein the indel is between 2-16 nucleotides.
4. The method of embodiment 2, wherein the polymorphism is a transgene.
5. The method of embodiment 2, wherein the polymorphism was produced through gene editing.
6. The method of embodiment 2, wherein the polymorphism was produced through gene recovery.
7. The method of embodiment 2, wherein the polymorphism was produced through cre/lox or flp/frt recombination.
8. The method of embodiment 2, wherein the polymorphism was produced through chemical mutagenesis.
9. The method of embodiment 2, wherein the genetic trait of interest was produced through transposable elements.
10. The method of embodiment 1, wherein the seed is selected from the group consisting of a forage crop, oilseed crop, grain crop, fruit crop, ornamental plants, vegetable crop, fiber crop, spice crop, nut crop, turf crop, sugar crop, tuber crop, root crop, and forest crop.
11. The method of embodiment 1, wherein the seed sample comprises corn, soybean, or sorghum.
12. The method of embodiment 1, wherein the genetic trait of interest comprises cytoplasmic male sterility, the dhurrin free trait, cannabinoid level, increased yield, herbicide tolerance, or pest resistance.
13. The method of embodiment 12, wherein the genetic trait of interest is pest resistance and the pest comprises a virus, insect, or bacterium.
14. The method of embodiment 1, wherein the genetic trait of interest comprises abiotic stress, drought, temperature, or salt content of the soil.
15. The method of embodiment 1, wherein the genetic trait of interest comprises a change in flavor, altered oil composition, altered protein composition, or altered carbohydrate composition.
16. The method of embodiment 15, wherein the genetic trait of interest is altered carbohydrate composition and the carbohydrate comprises a starch, sugar, or fiber.
17. The method of embodiment 1, wherein the genetic trait of interest is altered allergen or toxin level.
18. The method of embodiment 1, wherein the genetic trait of interest comprises altered plant architecture, altered time to flowering, sterility, or increased photosynthesis efficiency.
19. The method of embodiment 1, wherein the estimated quantitative level of trait purity is used for non-GMO certification.
20. A method of quantitative estimation of the level of a genetic trait within a seed sample by pyrosequencing comprising:
(a) acquiring at least one testing seed sample to be estimated for the level of a genetic trait of interest, a contaminant seed sample, and a pure seed sample which is pure for the genetic trait of interest;
(b) preparing seed standards by spiking the pure seed sample with various proportions of contaminant seed;
(c) growing the pure seed sample, contaminant seed sample, seed standards, and at the at least one testing seed sample;
(d) taking leaf punches and extracting genomic DNA from the pure seed sample, contaminant seed sample, seed standards, and the at least one testing seed sample;
(e) designing primers for amplification of the genomic region neighboring the genetic trait of interest and a sequencing primer;
(f) performing PCR amplification on the seed samples and seed standards using said primers;
(g) sequencing the amplicons on a pyrosequencer and calculating a regression equation using known trait purity values of seed standards and the allele frequency values given by the pyrosequencer; and
(h) calculating the estimated quantitative level of trait purity for the at least one testing seed sample using said regression equation.
21. The method of embodiment 1, wherein the genetic trait of interest is a stacked trait which comprises more than one polymorphism selected from the group consisting of SNPs, indels, and a variation in copy number.
22. A method of quantitative estimation of the level of a genetic trait within a seed sample by pyrosequencing comprising:
(a) acquiring at least one testing seed sample to be estimated for the level of a genetic trait of interest, a contaminant seed sample, and a pure seed sample which is pure for the genetic trait of interest;
(b) preparing seed standards by spiking the pure seed sample with various proportions of contaminant seed;
(c) extracting genomic DNA from the pure seed sample, contaminant seed sample, seed standards, and at the at least one testing seed sample;
(d) designing primers for amplification of the genomic region neighboring the genetic trait of interest and a sequencing primer;
(e) performing PCR amplification on the seed samples and seed standards using said primers;
(f) sequencing the amplicons through pyrosequencing and calculating a regression equation using known trait purity values of seed standards and the allele frequency values given by the pyrosequencer; and
(g) calculating the estimated quantitative level of trait purity for the at least one testing seed sample using said regression equation.
23. The method of embodiment 22, wherein the genetic trait of interest is a stacked trait which comprises more than one polymorphism selected from the group consisting of SNPs, indels, and a variation in copy number.
24. The method of embodiment 22, wherein the seed is selected from the group consisting of a forage crop, oilseed crop, grain crop, fruit crop, ornamental plants, vegetable crop, fiber crop, spice crop, nut crop, turf crop, sugar crop, tuber crop, root crop, and forest crop.
25. The method of embodiment 22, wherein the seed sample comprises corn, soybean, or sorghum.
26. The method of embodiment 22, wherein the genetic trait of interest comprises cytoplasmic male sterility, the dhurrin free trait, THC level, increased yield, herbicide tolerance, or pest resistance.
27. The method of embodiment 26, wherein the genetic trait of interest is pest resistance and the pest comprises a virus, insect or bacterium.
28. The method of embodiment 22, wherein the genetic trait of interest comprises abiotic stress, drought, temperature, or salt content of the soil.
29. The method of embodiment 22, wherein the genetic trait of interest comprises a change in flavor, altered oil composition, altered protein composition, or altered carbohydrate composition.
30. The method of embodiment 22, wherein the genetic trait of interest is altered carbohydrate composition and the carbohydrate comprises a starch, sugar, or fiber.
31. The method of embodiment 22, wherein the genetic trait of interest is altered allergen or toxin level.
32. The method of embodiment 22, wherein the genetic trait of interest comprises altered plant architecture, altered time to flowering, sterility, or increased photosynthesis efficiency.
33. The method of embodiment 22, wherein the estimated quantitative level of trait purity is used for non-GMO certification.
34. A method of quantitative estimation of the level of a genetic trait within a seed sample by next generation sequencing comprising:
(a) acquiring at least one testing seed sample to be estimated for the level of a genetic trait of interest, a contaminant seed sample, and a pure seed sample which is pure for the genetic trait of interest;
(b) preparing seed standards by spiking the pure seed sample with various proportions of contaminant seed;
(c) growing the pure seed sample, contaminant seed sample, seed standards, and at the at least one testing seed sample;
(d) taking leaf punches and extracting genomic DNA from the pure seed sample, contaminant seed sample, seed standards, and the at least one testing seed sample;
(e) designing primers for amplification of the genomic region neighboring the genetic trait of interest and a sequencing primer;
(f) performing PCR amplification on the seed samples and seed standards using said primers;
(g) sequencing the amplicons through next generation sequencing and calculating a regression equation using known trait purity values of seed standards and the allele frequency values given by the next generation sequencer; and
(h) calculating the estimated quantitative level of trait purity for the at least one testing seed sample using said regression equation.
35. A method of quantitative estimation of the level of a genetic trait within a seed sample by next generation sequencing comprising:
(a) acquiring at least one testing seed sample to be estimated for the level of a genetic trait of interest, a contaminant seed sample, and a pure seed sample which is pure for the genetic trait of interest;
(b) growing the pure seed sample, contaminant seed sample, and at the at least one testing seed sample;
(c) taking leaf punches and extracting genomic DNA from the pure seed sample, contaminant seed sample, and the at least one testing seed sample;
(d) preparing seed standards by spiking the pure seed sample genomic DNA extract with various proportions of contaminant seed genomic DNA extract;
(e) designing primers for amplification of the genomic region neighboring the genetic trait of interest and a sequencing primer;
(f) performing PCR amplification on the seed samples and seed standards using said primers;
(g) sequencing the amplicons through next generation sequencing and calculating a regression equation using known trait purity values of seed standards and the allele frequency values given by the next generation sequencer; and
(h) calculating the estimated quantitative level of trait purity for the at least one testing seed sample using said regression equation.
36. A method of quantitative estimation of the level of a genetic trait within a seed sample by next generation sequencing comprising:
(a) acquiring at least one testing seed sample to be estimated for the level of a genetic trait of interest, a contaminant seed sample, and a pure seed sample which is pure for the genetic trait of interest;
(b) extracting genomic DNA from the pure seed sample, contaminant seed sample, and at the at least one testing seed sample;
(c) preparing seed standards by spiking the pure seed sample genomic DNA extract with various proportions of contaminant seed DNA extract;
(d) designing primers for amplification of the genomic region neighboring the genetic trait of interest and a sequencing primer;
(e) performing PCR amplification on the seed samples and seed standards using said primers;
(f) sequencing the amplicons through next generation sequencing and calculating a regression equation using known trait purity values of seed standards and the allele frequency values given by the next generation sequencer; and
(g) calculating the estimated quantitative level of trait purity for the at least one testing seed sample using said regression equation.
37. The method of embodiment 36, wherein said genetic trait of interest comprises a polymorphism selected from the group consisting of SNPs, indels, and a variation in copy number.
38. The method of embodiment 37, wherein the indel is between 2-16 nucleotides.
39. The method of embodiment 37, wherein the polymorphism is a transgene.
40. The method of embodiment 37, wherein the polymorphism was produced through gene editing.
41. The method of embodiment 37, wherein the polymorphism was produced through gene recovery.
42. The method of embodiment 37, wherein the polymorphism was produced through cre/lox or flp/frt recombination.
43. The method of embodiment 37, wherein the polymorphism was produced through chemical mutagenesis.
44. The method of embodiment 37, wherein the genetic trait of interest was produced through transposable elements.
45. The method of embodiment 36, wherein the seed is selected from the group consisting of a forage crop, oilseed crop, grain crop, fruit crop, ornamental plants, vegetable crop, fiber crop, spice crop, nut crop, turf crop, sugar crop, tuber crop, root crop, and forest crop.
46. The method of embodiment 36, wherein the seed sample comprises corn, soybean, or sorghum.
47. The method of embodiment 36, wherein the genetic trait of interest comprises cytoplasmic male sterility, the dhurrin free trait, cannabinoid level, increased yield, herbicide tolerance, or pest resistance.
48. The method of embodiment 47, wherein the genetic trait of interest is pest resistance and the pest comprises a virus, insect, or bacterium.
49. The method of embodiment 36, wherein the genetic trait of interest comprises abiotic stress, drought, temperature, or salt content of the soil.
50. The method of embodiment 36, wherein the genetic trait of interest comprises a change in flavor, altered oil composition, altered protein composition, or altered carbohydrate composition.
51. The method of embodiment 50, wherein the genetic trait of interest is altered carbohydrate composition and the carbohydrate comprises a starch, sugar, or fiber.
52. The method of embodiment 36, wherein the genetic trait of interest is altered allergen or toxin level.
53. The method of embodiment 36, wherein the genetic trait of interest comprises altered plant architecture, altered time to flowering, sterility, or increased photosynthesis efficiency.
54. The method of embodiment 36, wherein the estimated quantitative level of trait purity is used for non-GMO certification.

These and/or other objects, features, advantages, aspects, and/or embodiments will become apparent to those skilled in the art after reviewing the following brief and detailed descriptions of the drawings. Furthermore, the present disclosure encompasses aspects and/or embodiments not expressly disclosed but which can be understood from a reading of the present disclosure, including at least: (a) combinations of disclosed aspects and/or embodiments and/or (b) reasonable modifications not shown or described.

### EXAMPLES

The examples provided below describe the application of pyrosequencing for estimating the trait genetic purity quantitatively by testing the genomic DNA from bulked seed and leaf tissues.

### Example 1: Genetic Purity Testing of Dhurrin Free Trait in Sorghum Seed Lots Using Pyrosequencing

Sorghum crop produces a cyanogenic glucoside, a secondary metabolite called Dhurrin. Dhurrin is toxic to animals when sorghum is used as a forage. Purdue University had developed a Sorghum type that does not produce Dhurrin (U.S. Pat. No. 9,512,437B2). In order to commercialize Dhurrin free Sorghum, a seed quality assessment method for assuring the dhurrin free trait genetic quality was required. Sorghum plants with dhurrin free trait have a Single Nucleotide Polymorphism (SNP) variation called as C493Y in the coding region of CYP79A1 gene (see U.S. Pat. No. 9,512,437B2, incorporated herein by reference).

Contaminants of sorghum seed lots with dhurrin free trait are the sorghum seed that make dhurrin (wild type allele). The assessment of percent sorghum seed that make dhurrin in each sorghum seed lot provides the genetic quality estimate for dhurrin free trait. In other words, low level or adventitious presence of sorghum seed that make dhurrin need to be estimated quantitatively. The goal of the trait providers was to give an assurance of 99% genetic purity of the trait. For detecting the low-level presence of contaminants at 95% confidence interval, at least 3000 seed need to be tested (Remund 2001). At DNA level, Dhurrin free sorghum differs from sorghum that makes dhurrin by a single base variation in CYP79A1 gene. Testing of 3000 seed individually using the available two assays; seedling-based Feigl-Anger assay, a biochemical method to check an individual seed's ability and RT-PCR based KASP genotyping technology for detecting SNP variation would be very expensive. Further, these methods are laborious, time consuming and expensive to practice on a production scale. Therefore, an alternative trait genetic quality testing method that is cheaper, faster, reliable and provides accurate assessment of trait genetic quality that could be applied on bulked seed would be valuable.

Allele frequency estimation of SNP genetic variation that differentiates dhurrin free trait from contaminants' genetic variation provides the quantitative estimate of trait genetic purity. For detecting and quantifying the adventitious presence of wild type SNP allele, since there is an in-house RT-PCR machine available, whether it could be used for quantitative estimation of adventitious presence was tested. The RT-PCR test determines what percent of the genomic DNA extracted from the representative sample of a seed lot has wild type specific SNP genetic variation when compared against known standards consisting of various levels of DNA from wild type and dhurrin free sorghum seed. This assay provides an indirect assessment of percent of wild type seed present in dhurrin free sorghum seed.

Good quality input DNA is a prerequisite for quantitative RT-PCR test for achieving accurate results and high detection sensitivity. Therefore, a method to extract genomic DNA from bulked seed that consistently yields good quality genomic DNA is critical. The chemistry of seed varies from species to species and often, it varies within a single species depending on the purpose for which a specific variety or hybrid is bred for. Therefore, DNA extraction method need to be optimized for each seed type for extracting good quality DNA. Previously developed methods for sorghum seed genomic DNA extraction yielded poor quality DNA and were time consuming. To overcome these limitations, a genomic DNA extraction method that is cheaper, faster and consistently yields good quality DNA for routine sorghum trait purity testing was developed.

Quantitative Real-Time PCR Assay Development for Dhurrin Free Trait

Primers: Primers were designed for amplification of the genomic region surrounding the SNP genetic variation. For a reliable quantitative assay, a 100±10% amplification efficiency of primers is necessary. For identifying an optimal primer pair with 100±10% amplification efficiency, four different forward, CYP79A1F, CYP79A1F2, CYP79A1F3, and CYP79A1F4 and three reverse, CYP79A1R, CYP79A1R2 and CYP79A1R3 primers were tested.

Allele specific Probe: CYP79Probe 1, a probe that is specific for the wild type SNP allele was designed.

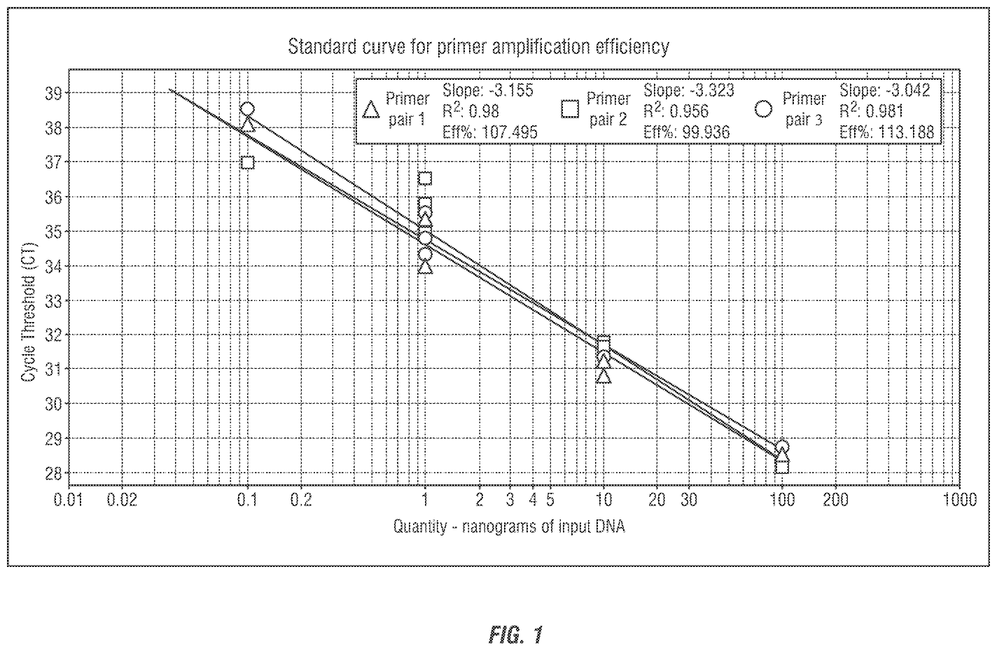

Identification of optimal primer and probe: Genomic DNA with wild type allele was used as template for testing the ability of the probe to bind and detect wild type allele and for assessing primer amplification efficiency. The primer pair 2, CYP79A1F and CYP79A1R3 was found to have efficient amplification of 99.99% when tested on DNA only with wild type allele (FIG. 1) in a 10-fold dilution series of 100, 10, 1, 0.1 and 0.01 nanograms per amplification reaction.

FIG. 1 illustrates a standard curve analysis for validating the amplification efficiency of three different primer pairs on RT-PCR; primer pair 1=CYP79A1F+CYP79R2; primer pair 2=CYP79A1F+CYP79R3; Primer pair 3=CYP79A1F2+CYP79R2 at 100, 10, 1, 0.1 and 0.01 ng of genomic DNA template from wild type seed.

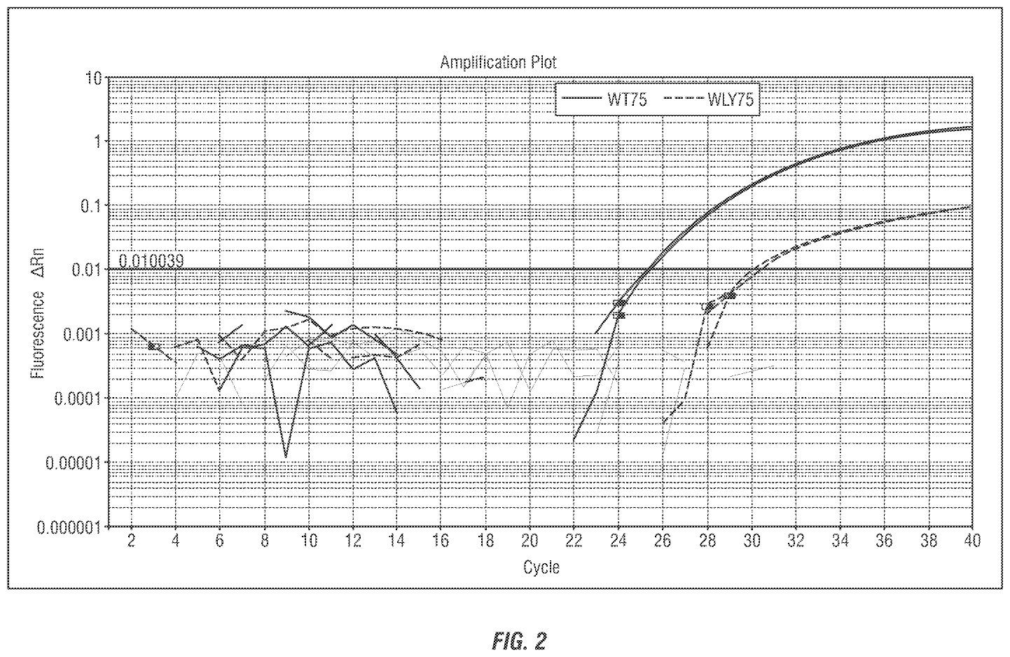

Detection limit of the probe: To further validate the specificity and the detection limit of the probe, various controls were tested. The controls were the DNA from Dhurrin free Sorghum seed (DNA with alternate SNP allele) and the Dhurrin free DNA spiked with various levels of wild type allele. Fluorescence was detected in the control with Dhurrin free DNA, indicating that the CYP79Probel was detecting both the wild type and dhurrin free DNA non-specifically (FIG. 2).

FIG. 2: Fluorescence was detected in PCR amplification from dhurrin free sorghum (WL75) DNA. The amplification plot shows the fluorescence from 75 nanograms of wild type (WT75) and dhurrin free (WL75) DNA.

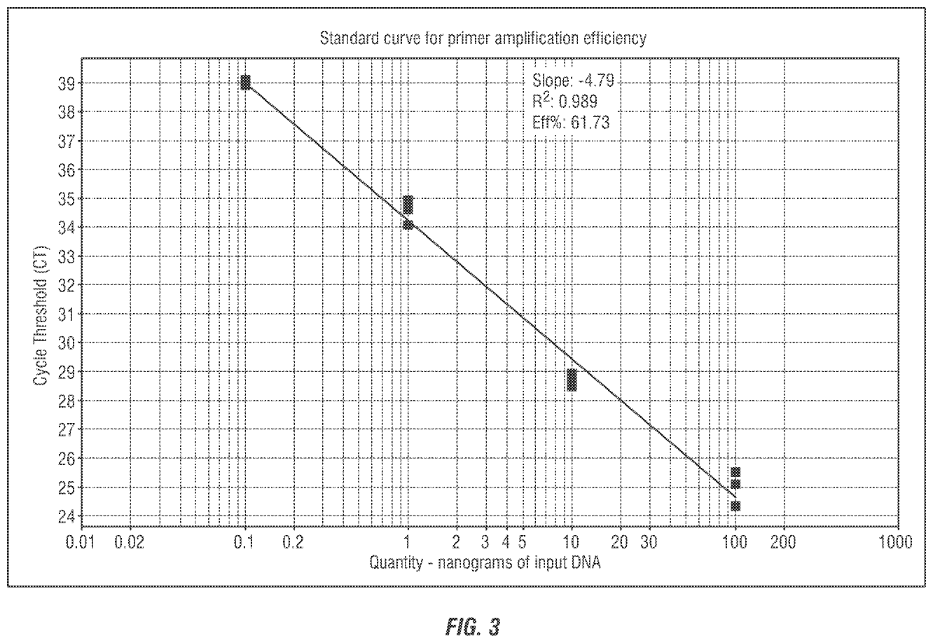

Improving the specificity of detection probe: Since the variation between the wild type and dhurrin free allele is only a single base pair, the chances are high that the probe can non-specifically bind to dhurrin free trait specific allele. To enhance the detection specificity of probe, a blocker oligo, which is complementary to dhurrin free trait specific SNP variation and the surrounding few bases was used in combination with primers designed to amplify only the wild type DNA. Further, another probe, CYP79Probe 2 was designed to improve the detection specificity. However, none of the primer pairs tested were proved to be efficient in PCR amplification. The amplification efficiency of primer pair CYP79A1ASPFR1 and CP79A1RASP1, CYP79Probe 2 probe with blocker Oligo is presented in FIG. 3.

FIG. 3 illustrates a standard curve analysis for validating the amplification efficiency of primer pair CYP79A1ASPFR1 and CP79A1RASP1 on RT-PCR with detection probe, CYP79Probe 2 at 100, 10, 1, 0.1 and 0.01 ng of genomic DNA template from wild type seed.

The quantitative RT-PCR assay was run on various test controls, including primer pair combinations, different genomic DNA template quantity and probe concentrations. However, reliable quantitative assay results could not be achieved.

Possible Cause of RT-PCR Method Failure

The SNP variation is present in a highly GC rich region (˜83% GC around the SNP site) and due to high GC content of the genomic region within 150 bps around the SNP, detection specificity of the probe could not be improved. Therefore, alternative methods needed to be identified.

Application of Pyrosequencing for Sorghum Dhurrin free trait quantitative trait genetic purity estimation: Since the Quantitative RT-PCR is not consistent in quantifying dhurrin free trait genetic purity, the applicability of real-time quantitative pyrosequencing-based method for reliability and accuracy of detection of adventitious presence of wild type sorghum seed or dhurrin free trait genetic purity was tested.

For testing this method, various controls were used, and initially, three blind samples were made by Ag Alumni Seed Improvement Association. The blind samples were made using hybrid seed of Tx623-C493Y, b6 X Excel-C493Y, tan, b6 from Summer 2016 production. Blind samples were made by mixing known quantity of wild type sorghum seed into dhurrin free seed. Blind samples were made based on 1000 seed weight. 1000 seed were weighed, and wild type seed were mixed in percent proportionate to 1000 seed weight. Two batches of seed produced in summer 2016 at two different locations were included in the genetic purity analysis. Genetic purity of dhurrin free trait for all the seed used for making standards was verified by using seedling-based assay.

Seedling based Feigl-Anger assay: During the development phase of dhurrin free Sorghum, a Purdue group used Feigl-Anger assay, a biochemical method to check an individual seed's ability to make dhurrin. The method uses the leaf tissue collected from a two-week-old seedling and looks for a blue spot on the Feigl-Anger paper after its exposure to HCN released from sorghum leaf tissue during a freeze thaw cycle. For determining the percent wild type seed (makes dhurrin) in a seed lot, seedlings can be tested as early as at 48 hours after imbibition. Trait purity for the seed lot of bmr6; C493Y using the seedling-based Feigl-Anger assay was 99.97%. Seed from this seed lot was used for making the spiked controls and blind samples. For every control and blind sample, three replicates of 1000 seed each were used for genomic DNA extraction. 1000 seed were taken based on 1000 seed weight. 1000 seed weight was calculated based on the seed weight of 10 replicates of 1000 seed counted manually.

Controls included:
1. Wild type Sorghum Seed
2. Dhurrin free Sorghum Seed
3. Dhurrin free Sorghum seed+0.1% wild type seed
4. Dhurrin free Sorghum seed+0.2% wild type seed
5. Dhurrin free Sorghum seed+0.5% wild type seed
6. Dhurrin free Sorghum seed+1.0% wild type seed
7. Dhurrin free Sorghum seed+2.0% wild type seed
8. Dhurrin free Sorghum seed+5.0% wild type seed

### Blind Samples

### 1. Entry 1

### 2. Entry 2

### 3. Entry 3

All the samples were ground using coffee grinder to a fine powder (10 seconds grinding each time for 4 times to be consistent across all samples and to get fine powder). 100 mg of this powder was used for genomic DNA extraction from all samples following the steps detailed below.

1. Added 1 ml of lysis buffer and 15 μl of Proteinase K (stored in −20° C. freezer) to each tube containing 100 mg of finely ground sorghum seed powder, mix thoroughly by vortexing for 2 minutes. Incubate in 60° C. water bath for 1 hour. Mix intermittently at about 30 minutes after incubation.
2. After incubation, centrifuged tubes @ 14000 rpm for 12 minutes. Transfer the supernatant (take only the clear lysate avoiding the cloudy top layer which could be protein) into a fresh tube.
3. Transfer the supernatant to a new tube. Add 5 μl of RNase A and incubate in 37° C. incubator for 1 hour for digesting residual RNA.
4. After digestion with RNase A, add 600-700 μl of 24:1 Chloroform: Iso Amyl Alcohol (if the supernatant is 400 add 400 μl of 24:1) and mix thoroughly by vortexing for about a minute. Centrifuge @ 10000 rpm for 15 minutes.
5. Repeated the Chloroform: Iso Amyl Alcohol extraction step.
6. Transferred the top liquid (˜250-300 μl) without touching the solid (ring like) middle layer to a new tube. Added half volume of 7.5M Ammonium acetate and 0.7× volume of Isopropanol (if the supernatant is 300 add 210 μl of Isopropanol). Mixed thoroughly and incubated at room temperature for 10 minutes.
7. Centrifuged @ 14000 rpm for 10 minutes. Poured off Isopropanol without losing the pellet. Washed the pellet by adding 800 μl of cold 70% ethanol. Inverted the tube several times to wash the pellet. Centrifuged @ 14000 rpm for 7 minutes. Removed ethanol and dried the pellet in 37° C. incubator for about 20 minutes.
8. Dissolved the pellet by adding 150 μl of TE buffer.

After genomic DNA extraction, DNA was checked for quality and quantity. Quality of DNA is considered good if the ratio of 260/280 is ˜1.8. The DNA was diluted to a 100 ng/μl final concentration. 50 ng (0.5 μl) of DNA was used for PCR

ICIA_F and ICIA_R primer pair was designed for amplifying the region surrounding the SNP variation. Reverse primer is 5′ biotinylated and HPLC purified for pyrosequencing purpose. The primers were ordered from IDT. Phusion hot start II polymerase kit from Thermo Fisher was used for PCR amplification of the marker.

### PCR Mix

| | |
|---|---|
| ddH₂O | 17.75 μl |
| GC Buffer | 5.0 μl |
| dNTPs | 0.5 μl |
| ICIA_F | 0.5 μl |
| ICIA_R | 0.5 μl |
| Genomic | 0.5 μl |
| DNA | |
| Phusion | 0.25 μl |
| Polymerase | |
| Total volume | 25.0 μl |

### PCR Cycler

conditions

98° C.—3 Minutes

| | | | |
|---|---|---|---|
| | 98° C.—10 Seconds | | |
| {open oversize brace} | 60° C.—10 Seconds | {close oversize brace} | 40X |
| | 72° C.—10 Seconds | | |

72° C.—5 minutes
4° C.—α

After PCR amplification of the SNP region, 1 micro liter of the amplicon from each well was tested on 1.0% agarose gel to check if the amplification has worked. Samples were shipped to Cincinnati Childrens' hospital's Pyrosequencing core facility for sequencing. Pyrosequencing was performed on PyroMark Q96 ID sequencing and quantification platform from Qiagen available from their website.

### Results

TABLE 1
Pyrosequencing results for the control and blind samples

| | | Pyrosequencer | |
|---|---|---|---|
| | | Allele | |
| Sample | | quantification % | Expected |
| ID | Genotype | G or A | % A |
| NTC | No Result | | 0.00 |
| WT | G/G | 98.70 | 0.00 |
| DF | A/A | 99.00 | 100.00 |
| Iso1 | A/A | 99.15 | 99.96 |
| Iso2 | A/A | 99.11 | 99.84 |
| 0.1 | A/A | 99.23 | 99.86 |
| 0.2 | A/A | 99.37 | 99.76 |
| 0.5 | A/A | 98.35 | 99.46 |
| 1 | A/A | 98.25 | 99.00 |
| 2 | A/A | 97.15 | 98.00 |
| 5 | A/A | 94.07 | 95.00 |
| E1 | A/A | 98.05 | Unknown |
| E2 | A/A | 95.80 | Unknown |
| E3 | A/A | 88.58 | Unknown |

WT = Wild Type alele—G;
DF = Dhurrin Free trait specific alele—A

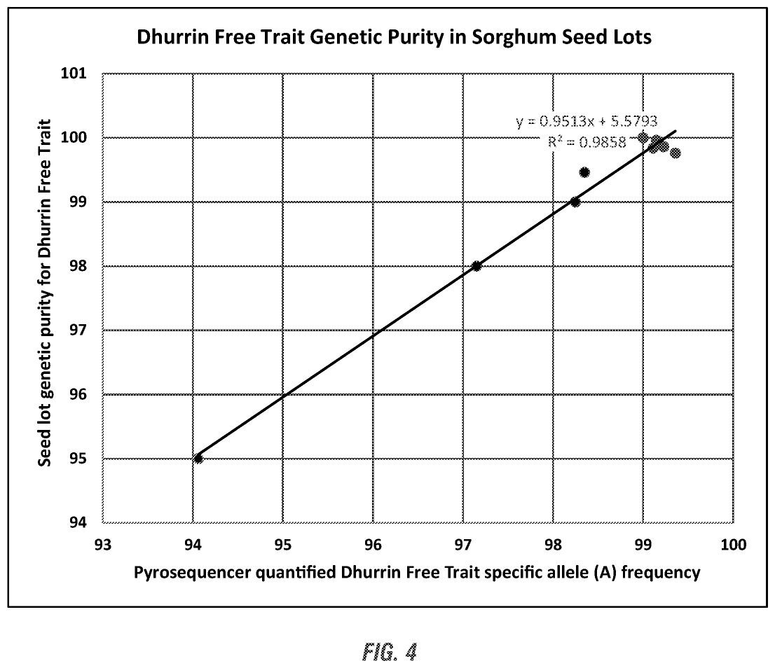

In FIG. 4, a regression equation is shown which was derived using the pyrosequencer estimated allele quantification values for the standards. The standards are the DNA extracted from spiked seed samples. Spiked seed samples were prepared by mixing known quantities of wild type seed (with no DF trait) to seed sample with dhurrin free trait. Spiked standards used were 0.1%, 0.2%, 0.3%, 0.5%, 1%, 2% and 5% wild type seed contamination. Regression equation was obtained by plotting pyrosequencer quantified allele frequency values from the spiked seed samples against the known spiking values (trait purity) and this regression equation was used for estimating the trait purity or level of contamination of unknown seed lots with sorghum seed consisting of wild type allele

Sequencing results were used for estimating the percent of seed with dhurrin free trait in a seed lot. The method estimates genetic purity of unknown samples using the pyrosequencer estimated allele quantification values in the regression equation derived from several DNA standards tested in every sequencing run. Based on the allele quantitation by sequencing values for G/A allele, the wild type contamination levels or DF Trait genetic purity for unknown (blind) samples, E1, E2 and E3 have been estimated. The estimated DF trait genetic purity for unknown samples, E1=98.81%, E2=96.68%, and E3=89.81%, closely match with the original values (samples were made by mixing WT seed into DF seed) of DF trait purity; E1=99%, E2=97%, E3=90%. The method was tested on more blind samples and in different genetic backgrounds to check if the method is repeatable and accurate in estimating the genetic purity. It was able to reliably estimate the purity of unknown samples, E4=99.377%, E5=97.3% and E6=85.3 and they all closely match with the actual values, E4=99.5%, E5=97%, and E6=85%. Trait genetic purity results from the pyrosequencing method and the actual values of trait purity were found to be strongly correlated, R²=0.996

Results obtained from several independent experiments accurately and reliably detected and quantified dhurrin free trait purity or the level of contamination of dhurrin free sorghum seed lots with sorghum seed that make dhurrin to as low as 0.5% contamination.

### Example 2: Corn CMS Fertile/Sterile Trait (SNP) Purity Testing Using Pyrosequencing

Applicability of the pyrosequencing method for estimating the genetic quality of other crop seed and traits was validated by testing corn seed for a trait with SNP genetic variation. In corn hybrid seed production, Cytoplasmic Male Sterility (CMS) trait is extensively used for cost effective hybrid corn seed production. There are several sources of CMS trait and based on the fertility ratings in various inbred backgrounds and the mitochondrial polypeptide variants specific for each type, male-sterile cytoplasm has been classified into three cytotypes, CMS-C, CMS-S and CMS-T, (Newton Kathleen J., 1988) whereas the fertile cytoplasm is classified into two cytotypes; NB and NA (Forde et al., n.d.; M-R Fauron & Casper, 1994). CMS cytotype CMS-T has not been in use in breeding programs due to its susceptibility to Southern Corn Leaf Blight. For differentiating the fertile and sterile cytotypes/cytoplasm, genetic variations of mitochondrial and plastid DNA are being used. Preference for CMS trait genetic purity varies depending on if the seed is used for seed or crop production. For hybrid seed production, seed of the female inbred line must be 100% pure for CMS trait and if the F1 hybrid seed is used for crop production, the preference for CMS trait purity varies from 30-60%.

Currently, at Indiana Crop Improvement Association (ICIA), genetic purity of CMS trait in a corn seed lot is tested by melt curve analysis on RT-PCR, which differentiates a mitochondrial SNP variation between fertile and sterile cytotypes. For each seed lot, RT-PCR assay tests DNA extracted from 90 individual seed and the trait purity for a seed lot is calculated based on genotype results from 90 individual RT-PCR assays. However, RT-PCR melt curve assay method is expensive to test 90 seed individually for every seed lot. Any method that could estimate trait purity by testing bulked seed would be valuable for reducing the time and cost for detecting CMS trait genetic purity.

The applicability of pyrosequencing method was tested for quantitative estimation of CMS trait genetic purity using DNA extracted from bulked seeds as well as bulked leaf punches. In order to detect and quantify the trait genetic purity, a genetic variation that differentiates both NA and NB fertile cytopes from CMS-C and CMS-S type sterile cytoplasm was identified by analyzing the mitochondrial and plastid genome sequences. A SNP genetic variation was identified in the coding region of InfA gene in the plastid genome of maize (Bosacchi et al., 2015).

A SNP (G/T) variation present within the coding sequence of InfA gene differentiates Both NB and NA type cytotypes from CMS-C and CMS-S cytotypes. Fertile cytotypes have G while sterile cytotypes have T at the same position. CMS-T plastid genome also has G at the SNP site. However, the CMS-T cytoplasm has not been in use in maize breeding due to its disease susceptibility. InfA F and InfA R primer pair was designed for amplifying the region surrounding the SNP variation.

Reverse primer is 5′ biotinylated and HPLC purified for pyrosequencing purpose. The primers were ordered from IDT.

For controls and blind samples, seed from seed lots for which trait purity was assessed as either 100% sterile or fertile in field grow out testing was requested from Beck's Hybrid Seed company, Atlanta, Indiana. Control seed standards and blind seed samples were made by mixing a proportion of sterile and fertile seed in percent seed weights. Control seed standards were made based on 1000 seed weight. 1000 seed weight was calculated based on the seed weight of 10 replicates of 1000 seed counted manually. For every control and blind sample, 2 replicates were used for genomic DNA extraction. For other samples, due to limited availability of seed, only 100 seed were used with no replications

Control seed standards included:

1. 100% sterile corn seed
2. 99% sterile corn seed+1% fertile corn seed
3. 95% sterile corn seed+5% fertile corn seed
4. 90% sterile corn seed+10% fertile corn seed
5. 80% sterile corn seed+20% fertile corn seed
6. 70% sterile corn seed+30% fertile corn seed
7. 60% sterile corn seed+40% fertile corn seed
8. 50% sterile corn seed+50% fertile corn seed
9. 40% sterile corn seed+60% fertile corn seed
10. 30% sterile corn seed+70% fertile corn seed
11. 20% sterile corn seed+80% fertile corn seed
12. 10% sterile corn seed+90% fertile corn seed
13. 100% fertile corn seed

Three blind samples, Blind1, Blind 2 and Blind 3 and 9 other samples, for which the genotypes for fertile/sterile trait were known by testing with melt curve assay on RT-PCR. These are Beck's blend, FR3, FR9, FR10, FR13, FR14, FR17, FR20 and FR36 were also included in the test. All the samples were ground using grinder to a fine powder. 100 mg of this powder was used for genomic DNA extraction from all samples

After genomic DNA extraction, DNA was verified for quality and quantity. Quality of DNA is considered good if the ratio of 260/280 is ˜1.8. The DNA was diluted to a 100 ng/μl final concentration. 100 ng (1.0 μl) of DNA was used for PCR. InfA F and InfA R forward and reverse primer pair was used for amplifying the region surrounding the SNP variation. Reverse primer is 5′ biotinylated and HPLC purified for pyrosequencing purpose. Phusion hot start II polymerase kit from Thermo Fisher was used for PCR amplification of the marker.

### PCR Mix

| | |
|---|---|
| ddH₂O | 17.25 μl |
| GC Buffer | 5.0 μl |
| dNTPs | 0.5 μl |
| ICIA_F | 0.5 μl |
| ICIA_R | 0.5 μl |
| Genomic | 1.0 μl |
| DNA | |
| Phusion | 0.25 μl |
| Polymerase | |
| Total volume | 25.0 ul |

### PCR Conditions

98° C.—3 minutes

| | | | |
|---|---|---|---|
| | 98° C.—10 Sec | | |
| {open oversize brace} | 57° C.—10 Sec | {close oversize brace} | 40X |
| | 72° C.—10 Sec | | |

72° C.—7 Min
4° C—∞

After PCR amplification of the SNP region, 1 micro liter of the amplicon from each well was tested on 1.0% agarose gel to check if the amplification has worked or not. Samples were shipped to two different Pyrosequencing service providers, Cincinnati Childrens' hospital's pyrosequencing core facility, Cincinnati, Ohio and EpigenDX, Hopkinton, Mass. (both use the same model of pyrosequencer as described in Example 1).

### Results

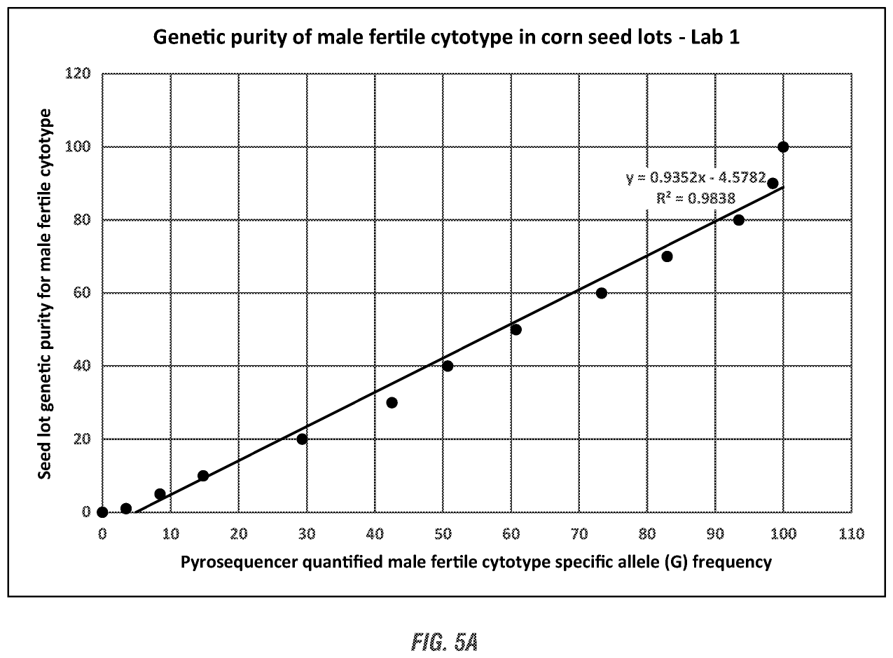

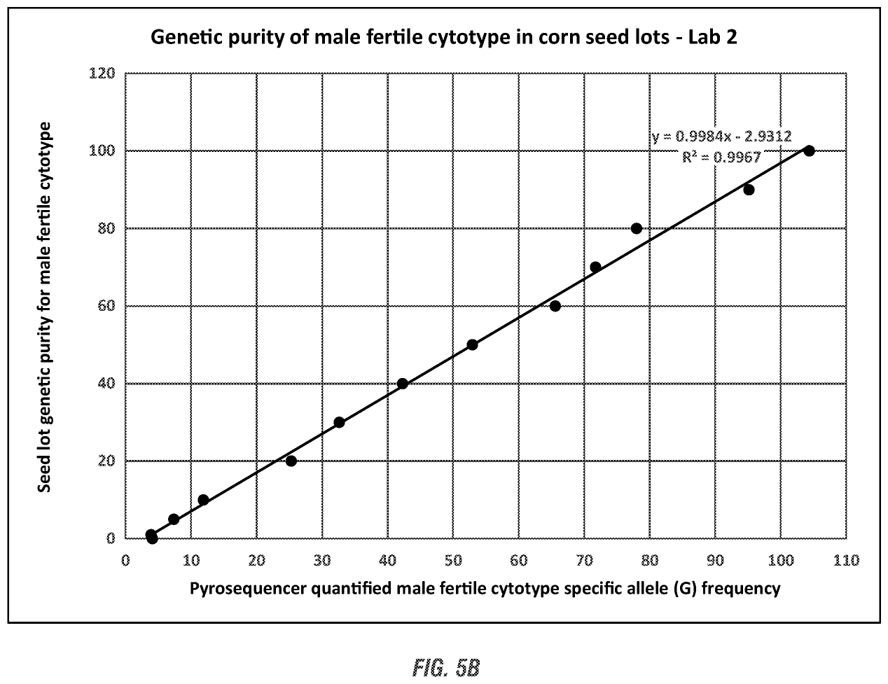

FIGS. 5A and 5B show regression equations derived using the pyrosequencer estimated allele quantification values for the control seed standards. The standards are the DNA extracted from spiked seed samples. Spiked seed samples were prepared by mixing known quantities of corn seed with cytoplasmic male sterile and fertile type seed. Spiked standards used were 99%, 95%, 90%, 80%, 70%, 60%, 50%, 40%, 30%, 20%, and 10% seed with sterile trait. Regression equation was obtained by plotting pyrosequencing results from the spiked seed samples against the known spiking values (trait purity) and this regression equation was used for estimating the trait purity or level of contamination of unknown seed lots.

TABLE 2
Comparison of different service providers and RT-PCR
melt curve assay with Fertile/sterile trait genetic purity
estimated from pyrosequencer quantified allele frequency.

| | Percent fertile seed | | |
|---|---|---|---|
| | RT-PCR | | |
| | Single seed | | |
| Sample | test based | Lab 1 | Lab 2 |
| FR3 | 75.28 | 77.62 | 71.50 |
| FR9 | 15.91 | 19.80 | 17.60 |
| FR10 | 1.12 | 0.00 | 0.00 |
| FR13 | 52.22 | 39.64 | 35.00 |
| FR14 | 34.44 | 39.08 | 34.40 |
| FR17 | 48.31 | 48.97 | 45.80 |
| FR20 | 61.11 | 64.80 | 59.80 |
| FR36 | 25.56 | 32.17 | 30.60 |
| Beck's blend | 54.50 | 38.00 | 33.00 |

TABLE 3
Fertile/sterile trait genetic purity estimated from pyrosequencer
quantified allele frequency for blind samples.

| | Percent fertile seed | | |
|---|---|---|---|
| | Percent | | |
| | fertile seed | | |
| Sample | spiked % | Lab 1 | Lab 2 |
| Blind 1 | 50.00 | 53.87 | 48.80 |
| Blind 2 | 20.00 | 23.30 | 20.10 |
| Blind 3 | 1.00 | 2.40 | 2.90 |

Pyrosequencing allele frequency values for control seed standards were used for deriving a regression equation for results from both service providers independently. Trait purity for various blind and other samples included in the study were calculated from the regression equation. Trait genetic purity or allele frequency results from the pyrosequencing method and RT-PCR melt curve assay were found to be strongly correlated, R²=0.883 for Lab 1 and R²=0.859 (Table 2). Trait purify estimates from pyrosequencing for the 3 blind samples also showed good correlation with actual purity for both Lab 1 and Lab 2 (Table 3), though the number of samples (3) were too small for meaningful statistical analysis.

These results from bulk seed testing were very encouraging. However, for some of the samples, the correlation of results between RT-PCR and Pyrosequencing methods was not as good as the rest of samples (Table 2), possibly due to variation in seed size produced by a fertile and male sterile corn plant. Weight of 1000 seed of corn produced on a male sterile female line is known to be higher (1.35×) and more variable when compared to the seed produced on a male fertile female line (Tabaković et al., 2017). To further improve purity estimate accuracy and to demonstrate that the disclosed invention also works with leaf samples, bulked leaf punches were used instead of bulked seed to conduct the experiment. Sterile and fertile seed were germinated on vermiculite-soil media. Leaf punches were collected from one-week old seedlings. A wide range of control standards were prepared by pooling a known number of leaf punches collected from sterile and fertile seed to a total of 100 punches (details provided below). For every control, two replicates were used for genomic DNA extraction.

Controls included:

1. 100% Sterile=100 leaf punches from male sterile seedlings
2. 100% Fertile=100 leaf punches from male fertile seedlings
3. 90% Fertile=90 Fertile+10 sterile

### 4. 80% Fertile=80 Fertile+20 Sterile

### 5. 75% Fertile=75 Fertile+25 Sterile

### 5. 70% Fertile=70 Fertile+30 Sterile

### 6. 60% Fertile=60 Fertile+40 Sterile

### 7. 50% Sterile=50 Fertile+50 Sterile

### 8. 40% Fertile=40 Fertile+60 Sterile

### 9. 30% Fertile=30 Fertile+70 Sterile

### 10. 20% Fertile=20 Fertile+80 Sterile

### 11. 10% Fertile=10 Fertile+90 Sterile

12. Blind sample 1
13. Blind sample 2
14. Blind sample 3
15. Blind sample 4
16. Blind sample 5

Five blind samples were prepared by a colleague by mixing known numbers of fertile and sterile corn seed to a total of 110 seed. Seeds of blind samples were planted, and leaf punches were collected from the germinated seed. Genomic DNA was extracted from bulked leaf punches. For control standards, leaf punches were collected in the proportion listed below.

Leaf punches were frozen in −80 C freezer. The frozen tissues were homogenized with pestle in 2 ml micro centrifuge tubes and further processed with CTAB method for genomic DNA extraction.

After genomic DNA extraction, DNA was verified for quality and quantity. Quality of DNA is considered good if the ratio of 260/280 is ˜1.8. The DNA was diluted to a 100 ng/μl final concentration. 100 ng (1.0 μl) of DNA was used for PCR. InfA F and InfA R forward and reverse primer pair was used for amplifying the region surrounding the SNP variation. Reverse primer is 5′ biotinylated and HPLC purified for pyrosequencing purpose. Phusion hot start II polymerase kit from Thermo Fisher was used for PCR amplification of the marker PCR mix

| | |
|---|---|
| ddH₂O | 17.25 μl |
| GC Buffer | 5.0 μl |
| dNTPs | 0.5 μl |
| ICIA_F | 0.5 μl |
| ICIA_R | 0.5 μl |
| Genomic | 1.0 μl |
| DNA | |
| Phusion | 0.25 μl |
| Polymerase | |
| Total volume | 25.0 ul |

### PCR Conditions

| 98° C.—3 minutes | | | |
|---|---|---|---|
| | 98° C.—10 Sec | | |
| {open oversize brace} | 57° C.—10 Sec | {close oversize brace} | 40X |
| | 72° C.—10 Sec | | |

72° C.—7 Min
40° C.—∞

After PCR amplification of the SNP region, 1 micro liter of the amplicon from each well was tested on 1.0% agarose gel to check if the amplification has worked or not. Samples were shipped to pyrosequencing service provider, EpigenDX, Hopkinton, Mass.

### Results

TABLE 4
Pyrosequencing results for the Control and blind
samples using bulk leaf bunches.

| | Pyrosequencer | |
|---|---|---|
| | Allele | |
| | quantification % | Expected |
| Sample ID | G | % G |
| NTC | | 0 |
| 100% Sterile | 3 | 0 |
| 100% Fertile | 102 | 100 |
| 90% Fertile | 89 | 90 |
| 80% Fertile | 79 | 80 |
| 75% Fertile | 68 | 75 |
| 70% Fertile | 61 | 70 |
| 60% Fertile | 66 | 60 |
| 50% Fertile | 45 | 50 |
| 40% Fertile | 40 | 40 |
| 30% Fertile | 27 | 30 |
| 25% Fertile | 19 | 25 |
| 20% Fertile | 17 | 20 |
| 10% Fertile | 18 | 10 |
| Blind 1 | 51.35 | Unknown |
| Blind 2 | 61.72 | Unknown |
| Blind 3 | 17.51 | Unknown |
| Blind 4 | 84.84 | Unknown |
| Blind 5 | 36.77 | Unknown |

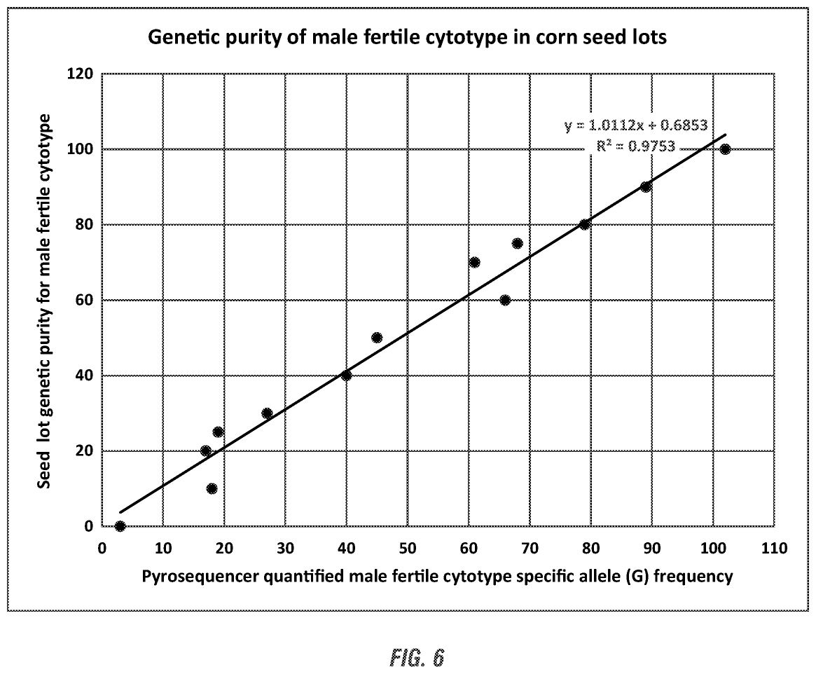

FIG. 6 illustrates a regression equation derived using the pyrosequencer quantified allele frequency values for the control standards made by pooling leaf punches in a known proportion, collected from seedlings of fertile and sterile cytotypes. X-axis: Male fertile cytotype specific ‘G’ allele frequency quantified by pyrosequencer. Y-axis: Genetic purity of male fertile cytotype. Standards used were 100%, 90%, 80%, 75%, 70%, 60%, 50%, 40%, 30%, 25%, 20%, 10% fertile cytotype and 100% sterile cytotypes. Regression equation was obtained by plotting pyrosequencer quantified fertile cytotype specific allele frequency values against the known spiked/trait purity values and this regression equation was used for estimating the trait purity for fertile cytotype or level of admixture of male sterile cytotypes of unknown seed lots.

TABLE 5
Fertile cytotype genetic purity estimated from pyrosequencer
quantified allele frequency for blind bulk leaf samples.

| | Percent fertile | Estimated |
|---|---|---|
| | cytotype seed | percent fertile |
| Unknown | (spiked %) | cytotype seed |
| Blind 1 | 50.90 | 52.54 |
| Blind 2 | 61.80 | 63.01 |
| Blind 3 | 15.50 | 18.36 |
| Blind 4 | 81.80 | 86.36 |
| Blind 5 | 38.20 | 37.81 |

Pyrosequencer quantified allele frequency values for control seed standards were used for deriving a regression equation. Trait purity for blind samples included in the study were calculated from the regression equation. Trait genetic purity or allele frequency results from the pyrosequencing method and known trait purity values for blind samples very strongly correlated using bulk leaf samples (R²=0.99, Table 5).

### Example 3 (Prophetic): Gene-Edited Trait Purity Testing Using Pyrosequencing

It is reasonable to expect that the current disclosed method can also be applied to determine trait purify for gene (genome)-edited traits in any crops or plants, provided the edit is a small nucleotide substitution (SNP for example) or small insertion/deletion (indel). DNA preparation, PCR amplification of DNA fragments surrounding the edited region and pyrosequencing will be the same as described in Examples 1 and 2. Gene-edited plant materials are very limited currently because very few gene-edited crops have been commercialized and almost all of them involved large DNA fragment deletion (gene knockout). However, that will change dramatically in the next few years as many startups and well-established agriculture companies as well as universities try to bring different gene-edited traits to the market.

### Example 4 (Prophetic): Stacked Trait Purity Testing Using Pyrosequencing

It is reasonable to expect that the current disclosed method can also be applied to determine trait purify for stacked traits in any crops or plants, provided the traits are caused by a small nucleotide substitution (SNP for example) or small insertion/deletion (indel). As more and more traits are identified (native) or created (Gene editing or GMO), it will be desirable to stack multiple beneficial traits in a single crop/plant variety, resulting the need for simultaneous determination of genetic purity of more than one trait. DNA preparation will be the same as described in Examples 1 and 2. PCR amplification of DNA fragments surrounding the edited region and pyrosequencing can be achieved in one of two approaches. In the first approach, PCR and pyrosequencing for multiple traits are done in uniplex, meaning all PCR and pyrosequencing reactions are done separately for each trait. In this approach, PCR and pyrosequencing procedures will be the same as described in Examples 1 and 2. In the second approach, PCR and pyrosequencing for multiple traits are done in multiplex to further reduce cost and turnaround time as described in (Ambroise et al. 2015).

### Example 5: Trait Purity Testing Using NextGen Sequencing

Next Generation Sequencing (NGS) technologies are those sequencing technologies that use massively parallel sequencing approach for nucleic acid sequencing. NGS technologies are high throughput, producing a high sequence data output in a short time at reduced cost. Based on the sequence read length, NGS technologies are further categorized as second generation short-read and third-generation real-time long-read technologies. Sequencing instruments from Illumina, Ion Torrent, BGI, ThermoFisher Scientific and Roche are short—read sequencers and PacBio and Nanopore's are of long-read sequencers. All sequencing platforms are based on sequencing by synthesis method except for BGI's, which uses sequencing by ligation method (Goodwin et al., 2016). Read length of short-read sequencing platforms varies from 36 bps to 600 bps depending on the sequencing chemistry used with a total sequence output ranging from 0.144 giga bases to 6,000 giga bases. For long-read sequencers, read length varies from 10 kilo bases to hundreds to thousands of kilo bases with a total sequence output ranging from 20 giga bases to 15,000 giga bases (Kumar et al., 2019).

NGS technologies have a wide variety of applications, including small genome sequencing, whole-genome sequencing, exome sequencing, whole transcriptome sequencing, targeted gene sequencing, gene expression profiling, RNA sequencing, methylation sequencing, miRNA and small RNA analysis and amplicon sequencing. In addition, multiple samples can be pooled (sample multiplexing) for sequencing, making NGS applicable for routine diagnostic testing. Though there are variations in sequencing strategy and chemistry, typical workflow for all NGS technologies involves three steps, sample preparation, sequencing, and data analysis (Goodwin et al., 2016).

The choice of NGS platform depends on the question that needs to be addressed, accessibility of sequencing platform, read length, read coverage, time, and the budget. NGS technologies have successfully been used for a variety of diagnostic applications (“First NGS-Based COVID-19 Diagnostic,” 2020, Hane Lee Julian A Martinez-Agosto Jessica Rexach Brent L Fogel, 2019; Yanchun Li, 2017) The methods disclosed in this invention could also be combined with any next generation sequencing (NGS) technologies with data analytics that could calculate the allele frequency information of either a specific locus or loci. PCR amplification will be the same as described in Examples 1 and 2 or modified according to different type of NGS requirements. Overall sequencing depth may need to be adjusted depending on the ranges of purity in the samples. Several different NGS technologies, including Illumina®, Roche 454, Ion torrent: Proton/PGM (ThermoFisher) and SOLiD (Applied BioSystems) were successfully used for estimating the trait genetic purity in the patent application WO PCT/EU2019/070386. The inventors divided the seed lots into several sublots and qualitative information of the sublots was used to derive the quantitative value of trait purity. More preferably, our disclosed invention could also be used in conjunction with BGI's DNBseq™ Technology: NGS 2.0, available on the BGI website. DNBseq™ Technology employs DNA NanoBalls platform that provides very high-density sequencing templates and increases higher Signal-to-Noise ratio; PCR-free Rolling-Circle Replication that makes only copies of the original DNA template instead of copy-of-a-copy and reduces sequencing errors. These and other unique features of DNBseg™ Technology has the potential to achieve higher sensitivity and accuracy in purity quantification, particularly in detecting low level of contaminants.

Genetic Purity Testing of Dhurrin Free Trait in Sorghum Seed Lots Using Illumina NextGen Sequencer MiSeq

Purdue University Genomics Core Facility recently launched a special sequencing service called WideSeq to address sequencing projects that require intermediate level of reads using NextGen Illumina sequencer MiSeq. We decided to test the WideSeq method using sorghum Dhurrin-free trait described in Example 1 above. We also modified the procedure for the preparation of control standards. Instead of spiking DF seeds with WT seeds to make a series of standards with different level of DF trait, we decided to extract DNA from 100% DF and 100% WT seeds separately and create a series of control DNA standards by spiking pure DF DNA with appropriate amount of WT DNA. This modification was made because we recently purchased a Qubit 4.0 Fluorometer that can accurately determine true DNA concentrations and spiking DNA has the potential to reduce variations and simplify overall procedures. More details are described below.

### DNA Extraction

Genomic DNA was extracted from 100% DF or 100% WT sorghum seed powder using the NucleoMag® DNA Food kit (Macherey-Nagel, Allentown, Pa.) according to the manufacturer's protocol. DNA was quantified using Qubit 4.0 Fluorometer (ThermoFisher Scientific, Waltham, Mass.) and both DF and WT sorghum DNA were diluted to 20 ng/μL. Control standard sample preparation

The control samples were prepared through DNA spiking to reach concentrations of 0.1%, 0.5%, 1.0%, 5.0%, 10.0%, 20.0%, 40.0%, 60%, 80.0%, and 90.0% of WT DNA contamination. Samples representing 100% DF and 100% WT sorghum DNA were also included.

1. 100% Dhurrin-free (DF) sorghum DNA
2. 100% Wild type (WT) sorghum DNA

### 3. 0.1% WT (99.9 μL DF DNA+0.1 μL WT DNA)

### 4. 0.5% WT (99.5 μl DF DNA+0.5 μl WT DNA)

### 5. 1% WT (99.0 μl DF DNA+1.0 μl WT DNA)

### 6. 5% WT (95.0 μl DF DNA+5 μl WT DNA)

### 7. 10% WT (90.0 μl DF DNA+10 μl WT DNA)

### 8. 20% WT (80.0 μl DF DNA+20 μl WT DNA)

### 9. 40% WT (60.0 μl DF DNA+40 μl WT DNA)

### 10. 60% WT (40.0 μl DF DNA+60 μl WT DNA)

### 11. 80% WT (20.0 μl DF DNA+80 μl WT DNA)

### 12. 90% WT (10.0 μl DF DNA+90 μl WT DNA)

### PCR Primers

In the pyrosequencing experiment described in Example 1, the amplicon was only 87 bp and the primers were located very closed to the functional SNP position. To better suited for NGS sequencing, new forward (ICIA_F2) and reverse (ICIA_R2) primers were designed spanning the region containing the functional SNP described in Example 1 to amply a larger fragment.

PCR Amplification and Gel Electrophoresis

The PCR reaction mix was prepared in a total volume of 25 μL containing 8.95 μL of sterile water, 12.5 μL of 2× Zymo reaction buffer, 0.5 μL of 10 mM dNTPs, 0.4 μL of 10 μM each forward and reverse primers, 2 μL of DNA template (20 ng/μL), and 0.25 μL of ZymoTaq™ DNA Polymerase (5U/μL) (Zymo Research). PCR amplification was performed with an initial denaturation of 5 min at 95° C. followed by 35 cycles of 30 sec denaturation at 95° C., 30 sec annealing at 65° C., and 20 sec extension at 72° C., with a final extension of 7 min at 72° C. The PCR was performed on three replications for each sample. Four μL of the amplification reaction from one replication of each sample was run on a 1.0% agarose gel to verify the presence of desired PCR products.

### WideSeq Sequencing Analysis

The PCR products were purified using the NucleoMag® NGS Clean-up and Size Select kit (Macherey-Nagel, Allentown, Pa.) according to the manufacturer's protocol and sent to the Genomics Core Facility at Purdue University, West Lafayette, Ind. for WideSeq sequencing analysis using Illumina's MiSeq. NGS library preparation and sequencing of each sample was performed individually according to the WideSeq protocol. The raw sequence reads were processed at the Purdue Genomics Core Facility and reads containing WT allele (G) and DF allele (A) were counted for each sample.

### Results

TABLE 6
The percentage of G or A quantified from the standard
controls using WideSeq sequencing analysis.

| | | Quantified % | | |
|---|---|---|---|---|
| Sample ID | Genotype | G | A | Known A % |
| DF | A/A | 0.167 | 99.833 | 100 |
| 0.1WT | G/A | 0.210 | 99.790 | 99.9 |
| 0.5WT | G/A | 0.603 | 99.397 | 99.5 |
| 1.0WT | G/A | 0.937 | 99.063 | 99 |
| 5.0WT | G/A | 3.936 | 96.064 | 95 |
| 10.0WT | G/A | 7.061 | 92.939 | 90 |
| 20.0WT | G/A | 15.224 | 84.776 | 80 |
| 40.0WT | G/A | 31.937 | 68.063 | 60 |
| 60.0WT | G/A | 49.556 | 50.444 | 40 |
| 80.0WT | G/A | 68.598 | 31.402 | 20 |
| 90.0WT | G/A | 82.713 | 17.287 | 10 |
| WT | G/G | 99.711 | 0.289 | 0 |

DF = Dhurrin-free trait specific allele—A,
WT = Wild-type allele—G

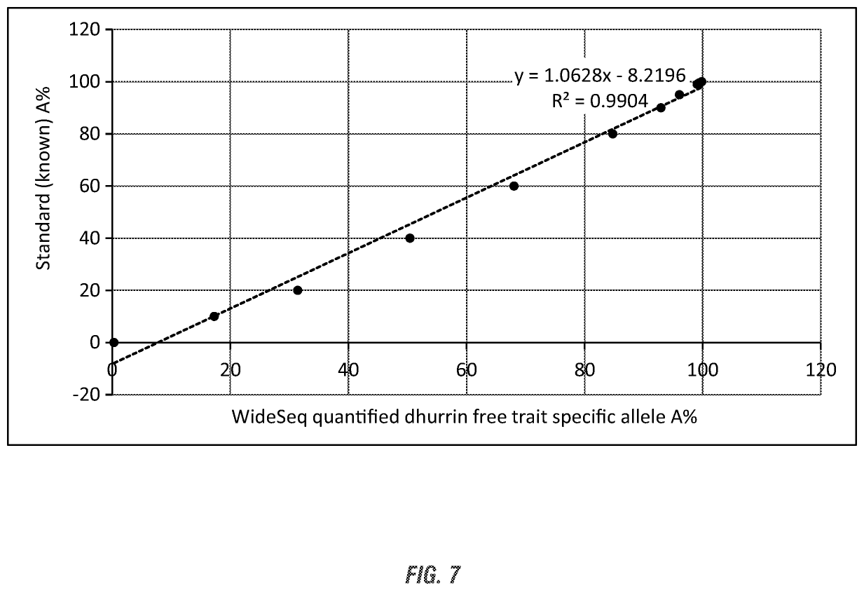

FIG. 7 illustrates a linear regression equation derived from the WideSeq estimated allele quantification values obtained from the standard samples. The standards were prepared by spiking DNA of known concentration. Linear regression equation was obtained by plotting WideSeq quantified allele frequency values from the standard samples against the known spiking values (trait purity). This linear regression equation can be used for estimating the trait purity or level of contamination of unknown seed lots with sorghum seed consisting of wild type allele when DNA are extracted from such seed lots and subjected to NextGen Sequencing using MiSeq.

As shown in FIG. 7, the estimated trait genetic purity results from the WideSeq sequencing and the known values of trait purity were found to be strongly correlated (R²=0.9904) in a series of control standards. The method can be used to estimates genetic purity of unknown samples using WideSeq estimated allele quantification values in the linear regression equation derived from several DNA standards tested in every sequencing run.

Therefore, we have now demonstrated that NextGen sequencing method WideSeq using Illumina's MiSeq instrument can accurately estimate the trait purify for sorghum DF trait. The results also show that spiking DNA can also be used to effectively generate a series of control standards.

### Discussion

Pyrosequencer detects and quantifies the genetic variation by sequencing amplicons with a sequencing primer binding at the intersection of the site of genetic variation that differentiates the contaminant and the desired trait. The approach of the use of several DNA standards containing known proportions of desired target and contaminant DNA helps in accurately assessing the trait genetic purity of an unknown seed lot over a wide range. The number of standards and proportion of a contaminant in a standard can be varied according to the requirements for the purity of a given trait.

Based on our results, the detection sensitivity (lower limit of detection) of the assay for seed lot contamination with seed of unwanted traits was 0.5% (Sorghum dhurrin free trait) and accurately assessed the purity of a trait over a wide range of contamination. Applicability of the method for estimating the genetic quality of other crop seed and traits was verified by testing corn seed for a trait with SNP genetic variation and satisfactory results were obtained for the tested trait. In principle, this method could be applied to genetic purity testing of both native and gene edited traits with various types of genetic variation, including SNP variation, few base pair insertion and deletion variation in a bulked seed sample.

Currently, various methods are used for seed/trait genetic purity estimation of a seed lot depending on the genetic, physiological, developmental and biochemical nature of the trait. However, Except the RT-PCR method, all methods are dependent on individual seed testing of 30-400 seed for providing quantitative estimate using the qualitative, presence/absence information obtained from single seed testing.

RT-PCR is routinely used for detecting and quantifying the admixture/adventitious presence of genetically engineered crops (GMO) in conventional seed lots and food supply chain. For the detection and quantitation of GMO contamination or trait genetic purity, RT-PCR method amplifies a DNA region with genetic variation and uses a fluorescent probe made up of DNA sequences complementary to the genetic variation of unwanted genetic trait within the amplicon. The fluorescence emitted by the probe upon its binding to the complementary DNA sequence is used for estimating the level of contamination either by comparing against a set of reference standards or using an endogenous gene. The accuracy and reliability of RT-PCR method depends on several factors:

1. Nature of genetic variation: Single base pair genetic variation is difficult to be quantified since the variation is only a single base pair, probe binds all complementary DNA sequences including the complementary DNA sequence with a SNP variation that corresponds to desirable trait. Due to this reason, RT-PCR method could not be applied for genetic purity estimation of traits and seed lots with SNP variation.
2. The location of genetic variation: DNA sequence composition adjacent to the site of genetic variation influences amplicon and probe chemistry and sensitivity of detection
3. Requires high quality input DNA. Chemistry and composition of each type of seed is different, requiring the development of DNA extraction protocol for good quality DNA for each seed type (Alarcon et al., 2019)
4. Amplification efficiency of PCR primers affects detection accuracy. Requires designing and testing of several primer pairs to achieve optimal amplification efficiency
5. Amount of probe used for detection needs to be standardized. Further, the specificity of the detection probe used in RT-PCR based-detection method is affected by the nature of genetic variation, more specifically, Single Nucleotide Polymorphism and insertion/deletion variations of few base pairs.
6. Though it has an excellent lower detection limit of 0.01%, the range for upper limit of detection is very narrow. RT-PCR, when used for testing the trait purity on a bulked seed sample, is not able to differentiate between 99 and 95% purity (Alarcon et al., 2019). Depending on the chemistry of the DNA sequence tested, the upper limit of detection varies from 5% to 50% (Chandra-Shekara et al., 2011)

Technically, any next generation sequencing technology could be applied for testing the trait genetic purity of a seed lot. The methods described in WO PCT/EU2019/070386 where NGS was used for assessing the trait genetic purity, the seed lots were divided into several sublots and qualitative information of the sublots was used for deriving the quantitative value of trait purity.

One of the NextGen Sequencing methods, Wide Seq using Illumina's MiSeq instrument, was also used to demonstrate that NextGen sequencing can accurately estimate the trait purify for sorghum DF trait (FIG. 7).

When compared to RT-PCR, the assay development for the pyrosequencing method or NextGen sequencing are faster and could be the only options available for bulked seed testing for detecting and quantifying purity of traits and seed lots with SNP genetic variation.

Other merits of the pyrosequencing or NextGen sequencing method over RT-PCR include:

1. Quality of input DNA and amplification efficiency of PCR primers do not affect sequencing
2. Probe design and standardization are not required
3. Nature and location of genetic variation does not affect the sensitivity of detection.
4. Has a broad range of Limit of Detection (LOD) and Limit of Quantification (LOQ) of 0.5 to 99.5%
5. NextGen sequencing methods also allow multiplexing, and therefore the ability to determine trait purity of multiple traits simultaneously and at a lower cost.

In summary, the above non-limiting examples are provided using either pyrosequencing or NextGen sequencing (MiSeq specifically) methods to determine trait genetic purity in Sorghum DF trait or corn CMS fertile/sterile trait. Furthermore, these methods are illustrated as effective whether the linear regression equations used to calculate the trait purity of unknown samples are derived from a series of control samples created by spiking seeds, leaf tissues or extracted DNA.

### REFERENCES

Alarcon, C. M., Shan, G., Layton, D. T., Bell, T. A., Whipkey, S., & Shillito, R. D. (2019). Application of DNA- and Protein-Based Detection Methods in Agricultural Biotechnology. Journal of Agricultural and Food Chemistry, 67(4), 1019-1028.
Bosacchi, M., Gurdon, C., & Maliga, P. (2015). Plastid genotyping reveals the uniformity of cytoplasmic male sterile-T maize cytoplasms. Plant Physiology, 169(3), 2129-2137.
Cankar, K., Stebih, D., Dreo, T., Žel, J., & Gruden, K. (2006). Critical points of DNA quantification by real-time PCR—Effects of DNA extraction method and sample matrix on quantification of genetically modified organisms. BMC Biotechnology, 6.
Chandra-Shekara, A. C., Pegadaraju, V., Thompson, M., Vellekson, D., & Schultz, Q. (2011). A novel DNA-based diagnostic test for the detection of annual and intermediate ryegrass contamination in perennial ryegrass. Molecular Breeding, 28(2), 217-225.
Chen J, Z. C. O. N. P. C. F. J. (2016). The Development of Quality Control Genotyping Approaches: A Case Study Using Elite Maize Lines. PLOS ONE, 11(6), e0157236.
Das, M. K., Ehrlich, K. C., & Cotty, P. J. (2008). Use of pyrosequencing to quantify incidence of a specific Aspergillus flavus strain within complex fungal communities associated with commercial cotton crops. Phytopathology, 98(3), 282-288.
Demeke, T., & Jenkins, G. R. (2010). Influence of DNA extraction methods, PCR inhibitors and quantification methods on real-time PCR assay of biotechnology-derived traits. In Analytical and Bioanalytical Chemistry (Vol. 396, Issue 6, pp. 1977-1990).
El-Deiry, W. S., Goldberg, R. M., Lenz, H., Shields, A. F., Gibney, G. T., Tan, A. R., Brown, J., Eisenberg, B., Heath, E. I., Phuphanich, S., Kim, E., Brenner, A. J., & Marshall, J. L. (2019). The current state of molecular testing in the treatment of patients with solid tumors, 2019. CA: A Cancer Journal for Clinicians.
First NGS-based COVID-19 diagnostic. (2020). In Nature biotechnology (Vol. 38, Issue 7, p. 777). NLM (Medline).
Forde, B. G., Oliver, R. J. C., Leaver, C. J., Gunn, R. E., & Kemblet, R. J. (n.d.). CLASSIFICATION OF NORMAL AND MALE-STERILE CYTOPLASMS IN MAIZE. I. ELECTROPHORETIC ANALYSIS OF VARIATION IN MITOCHONDRIALLY SYNTHESIZED PROTEINS.
Goodwin, S., McPherson, J. D., & McCombie, W. R. (2016). Coming of age: Ten years of next-generation sequencing technologies. In Nature Reviews Genetics (Vol. 17, Issue 6, pp. 333-351). Nature Publishing Group.
Gowda, M., Worku, M., Nair, S. K., Palacios-Rojas, N., Huestis, G., & Prasanna, B. M. (n.d.). Quality Assurance/Quality Control (QA/QC) in Maize Breeding and Seed Production: Theory and Practice.
Hane Lee Julian A Martinez-Agosto Jessica Rexach Brent L Fogel. (2019). Next Generation Sequencing in clinical diagnosis. Lancet Neurology, 18(5), 426-undefined.
Holst-Jensen, A., Rønning, S. B., Løvseth, A., & Berdal, K. G. (2003). PCR technology for screening and quantification of genetically modified organisms (GMOs). Analytical and Bioanalytical Chemistry, 375(8), 985-993.
Kumar, K. R., Cowley, M. J., & Davis, R. L. (2019). Next-Generation Sequencing and Emerging Technologies. Seminars in Thrombosis and Hemostasis, 45(7), 661-673.
Laffont, J.-L., Remund, K. M., Wright, D., Simpson, R. D., & Grégoire, S. (2005). Testing for adventitious presence of transgenic material in conventional seed or grain lots using quantitative laboratory methods: statistical procedures and their implementation. Seed Science Research, 15(3), 197-204.
M-R Fauron, C., & Casper, M. (1994). A Second Type of Normal Maize Mitochondrial Genome: An Evolutionary Link.
Newton Kathleen J. (1988). PLANT MITOCHONDRIAL GENOMES: ORGANIZATION, EXPRESSION AND VARIATION. Annual Review of Plant Physiology and Plant Molecular Biology, 39, 503-532.
Remund, K. M., Dixon, D. A., Wright, D. L., & Holden, L. R. (2001). Statistical considerations in seed purity testing for transgenic traits. Seed Science Research, 11(2), 101-120.
Smith, J. S. C., & Register III, J. C. (1998). Genetic purity and testing technologies for seed quality: a company perspective. Seed Science Research, 8(2), 285-294.
Song, Q., Wei, G., & Zhou, G. (2014). Analysis of genetically modified organisms by pyrosequencing on a portable photodiode-based bioluminescence sequencer. Food Chemistry, 154, 78-83.
Tabaković, M., Stanisavljević, R., Štrbanović, R., Poštić, D., & Kulić, G. (2017). VARIABILITY OF SEED TRAITS OF FERTILE AND STERILE VARIANTS OF THE MAIZE HYBRID COMBINATION ZP 434 VARIJABILNOST OSOBINA SEMENA FERTILNE I STERILNE VARIJANTE HIBRIDNE KOMBINACIJE KUKURUZA ZP 434. In Journal on Processing and Energy in Agriculture (Vol. 21).
Tsiatis, A. C., Norris-Kirby, A., Rich, R. G., Hafez, M. J., Gocke, C. D., Eshleman, J. R., & Murphy, K. M. (2010). Comparison of Sanger sequencing, pyrosequencing, and melting curve analysis for the detection of KRAS mutations: Diagnostic and clinical implications. Journal of Molecular Diagnostics, 12(4), 425-432.
Yanchun Li, J. G. J. S. D. B. K. L. M. J. P. K. K. V. B. K. S. B. (2017). Ion Torrent™ Next Generation Sequencing—Detect 0.1% Low Frequency Somatic Variants and Copy Number Variations simultaneously in Cell-Free DNA.

We claim:

1. A method of quantitative determination of the level of a genetic trait within a seed sample by pyrosequencing or next generation sequencing comprising:
(a) acquiring at least one testing seed sample to be estimated for the level of a genetic trait of interest, a contaminant seed sample, and a pure seed sample which is pure for the genetic trait of interest;
(b) preparing standards by spiking the pure seed samples with various proportions of contaminant seed;
(c) extracting genomic DNA from the pure seed sample, contaminant seed sample, seed standards, and at the at least one testing seed sample;
(d) designing primers for amplification of the genomic region neighboring the genetic trait of interest and a sequencing primer;
(e) performing PCR amplification on the seed samples and seed standards using said primers;
(f) sequencing the amplicons on a pyrosequencer or through next generation sequencing and calculating a regression equation using known trait purity values of seed standards and the allele frequency values given by the pyrosequencer or next generation sequencing; and
(g) calculating the estimated quantitative level of trait purity for the at least one testing seed sample using said regression equation.

2. The method of claim 1, wherein said genetic trait of interest comprises a polymorphism selected from the group consisting of SNPs, indels, and a variation in copy number.

3. The method of claim 2, wherein the polymorphism is a transgene.

4. The method of claim 2, wherein the polymorphism was produced through gene editing.

5. The method of claim 2, wherein the polymorphism was produced through gene recovery.

6. The method of claim 1, wherein the seed is selected from the group consisting of a forage crop, oilseed crop, grain crop, fruit crop, ornamental plants, vegetable crop, fiber crop, spice crop, nut crop, turf crop, sugar crop, tuber crop, root crop, and forest crop.

7. The method of claim 1, wherein the seed sample is corn.

8. The method of claim 1, wherein the seed sample is soybean.

9. The method of claim 1, wherein the seed sample is sorghum.

10. The method of claim 1, wherein the genetic trait of interest is cytoplasmic male sterility.

11. The method of claim 1, wherein the genetic trait of interest is the dhurrin free trait.

12. The method of claim 1, wherein the genetic trait of interest is cannabinoid level.

13. The method of claim 1, wherein the genetic trait of interest in increased yield.

14. The method of claim 1, wherein the genetic trait of interest is herbicide tolerance.

15. The method of claim 1, wherein the genetic trait of interest is pest resistance.

16. The method of claim 1, wherein the genetic trait of interest is abiotic stress.

17. The method of claim 1, wherein the genetic trait of interest is a stacked trait which comprises more than one polymorphism selected from the group consisting of SNPs, indels, and a variation in copy number.

18. The method of claim 1, wherein the estimated quantitative level of trait purity is used for non-GMO certification.

19. A method of quantitative estimation of the level of a genetic trait within a seed sample by next generation sequencing comprising:
(a) acquiring at least one testing seed sample to be estimated for the level of a genetic trait of interest, a contaminant seed sample, and a pure seed sample which is pure for the genetic trait of interest;
(b) preparing seed standards by spiking the pure seed sample with various proportions of contaminant seed;
(c) growing the pure seed sample, contaminant seed sample, seed standards, and at the at least one testing seed sample;
(d) taking leaf punches and extracting genomic DNA from the pure seed sample, contaminant seed sample, seed standards, and the at least one testing seed sample;
(e) designing primers for amplification of the genomic region neighboring the genetic trait of interest and a sequencing primer;
(f) performing PCR amplification on the seed samples and seed standards using said primers;
(g) sequencing the amplicons through next generation sequencing and calculating a regression equation using known trait purity values of seed standards and the allele frequency values given by the next generation sequencer; and
(h) calculating the estimated quantitative level of trait purity for the at least one testing seed sample using said regression equation.

20. A method of quantitative estimation of the level of a genetic trait within a seed sample by next generation sequencing comprising:
(a) acquiring at least one testing seed sample to be estimated for the level of a genetic trait of interest, a contaminant seed sample, and a pure seed sample which is pure for the genetic trait of interest;
(b) extracting genomic DNA from the pure seed sample, contaminant seed sample, and at the at least one testing seed sample;
(c) preparing seed standards by spiking the pure seed sample genomic DNA extract with various proportions of contaminant seed DNA extract;
(d) designing primers for amplification of the genomic region neighboring the genetic trait of interest and a sequencing primer;
(e) performing PCR amplification on the seed samples and seed standards using said primers;
(f) sequencing the amplicons through next generation sequencing and calculating a regression equation using known trait purity values of seed standards and the allele frequency values given by the next generation sequencer; and
(g) calculating the estimated quantitative level of trait purity for the at least one testing seed sample using said regression equation.
